(19) United States
(12) Patent Application Publication
(10) Pub. No.: US 2009/0312358 A1
(43) Pub. Date: Dec. 17, 2009

(54) METHOD FOR MANAGEMENT OF DIARRHEA

(75) Inventor: Fouad A. Siddiqi, Brookline, MA (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, Boston, MA (US)

(21) Appl. No.: 12/294,003

(22) PCT Filed: Mar. 22, 2007
(86) PCT No.: PCT/US07/07126

§ 371 Date: Feb. 23, 2009

Related U.S. Application Data
(60) Provisional application No. 60/784,661, filed on Mar. 22, 2006.

(51) Int. Cl. A61K 31/485; A61K 31/445; A61K 31/451; A61K 31/222; A61P 1/12

(52) U.S. Cl. 514/282; 514/317; 514/327; 514/330; 514/331; 514/513

(57) ABSTRACT

The present invention is directed to methods of treatment and/or management of diarrhea, such as chronic diarrhea using sequential administration of opioid agonists to suppress gut mobility and opioid antagonists to reverse the effect to controllably allow bowel movements. The agonists and antagonists are administered with a time interval in between the administration or between the release of the drugs from a pharmaceutical composition. The invention is further directed to methods of controlling, treating or managing side effects caused by the opioid agonists, specifically the side effects resulting from mast cell activation and/or granulation.

### CROSS-REFERENCE TO RELATED APPLICATIONS

This application is a National Phase Entry Application under 35 U.S.C. §371 of co-pending International Application PCT/US2007/007126, filed Sep. 29, 2006, which claims benefit under 35 U.S.C. §119(e) of provisional application Ser. No. 60/784,661, filed Mar. 22, 2007, the content of which is herein incorporated by reference in its entirety.

### BACKGROUND OF THE INVENTION

1. Field of the Invention

The present invention relates to methods for treatment and management of diarrhea, especially diarrhea due to diseases characterized by intestinal inflammation. The invention also relates to methods for treatment and management of diarrhea due to diseases characterized by intestinal inflammation, mast cell hyperplasia, or mast cell degranulation. The invention further relates to methods for alleviating side effects caused by antidiarrheal treatments.

2. Background of the Invention

Inflammatory bowel disease (IBD) is a chronic inflammatory disease of the intestine of unknown etiology. IBD has conventionally been divided into two broad subtypes, Crohn's disease (CD), and ulcerative colitis (UC). Ulcerative colitis is typically characterized by mucosal inflammation and ulceration that is limited to the colon; in Crohn's disease, transmural inflammation and ulceration can occur anywhere along the entire gastrointestinal tract. IBD is a lifelong, relapsing illness, with patients typically experiencing alternating periods of active disease and remission. Inflammatory bowel disease is estimated to affect at least one million people in the United States and an additional one million people in Europe (Botoman et al., 1998; Farrell et al., 2002; Kornbluth et al., 2004; Brunton et al., 2005).

One of the most prominent symptoms of active inflammatory bowel disease is profuse, chronic bloody diarrhea. Other symptoms include weight loss, anemia, abdominal pain, rectal urgency, cramping, and fever. The diarrhea caused by IBD is frequent, highly unpredictable and is usually accompanied by some degree of fecal incontinence. Patients with severe disease may have more than 20 bowel movements per day (Hendrickson et al., 2002; Carter et al., 2004).

Chronic, uncontrollable diarrhea is financially and socially devastating to patients (Fine et al., 1999; Cooper et al., 2000; Rao 2004). This aspect of IBD, beyond being incapacitating, can be embarrassing and humiliating, and severely degrades the quality of life of patients (Wingate et al., 2001; Smith et al., 2002; Carter et al., 2004; Rutgeerts et al., 2004). Patients with IBD tend to miss more workdays than do individuals without the disease, and some may not be able to maintain employment at all (Lichtenstien et al., 2003). Parents who have IBD report that chronic diarrhea has severe adverse effects on their ability to care for their children (Mukherjee et al., 2002). Chronic diarrhea is recognized as a major factor contributing to disability caused by IBD (Palmer et al., 1980; Fine et al., 1999; Smith et al., 2002; Scarlett, 2004).

Diarrhea in inflammatory bowel disease may persist until the patient goes into remission, which may be months or years, if at all. None of the opioid antidiarrheals are well-suited for long-term use in IBD. Daily dosing with these agents tends to produce severe side effects such as tenesmus, rectal urgency, nausea, cramping, abdominal pain, and bloating. These side effects may persist for several days after antidiarrheal treatment is withdrawn (Kornbluth et al., 2004; Rao 2004). These side effects occur because of reduced intestinal motility and resolve once full baseline motility has returned. In long-term use, morphine and codeine are addictive, diphenoxylate/difenoxin has a high incidence of CNS side effects, and loperamide tends to be too potent (Scarlett 2004; Tuteja et al., 2004).

Tolerance to the antimotility and antisecretory effects of most opioids develops very slowly (Foss 2001; Pappagallo 2001; Brunton et al., 2005), but tolerance after repeated dosing may be a serious problem for IBD patients since IBD is a chronic, lifelong disease. Tolerance to the antimotility effects of loperamide develops after only two days of repeated dosing in mice, but tolerance to the antimotility effects of morphine is not observed under the same conditions (Tan-No et al., 2003). Tolerance to loperamide is of particular concern, since many IBD patients will have had significant exposure to this drug even before they are aware that they have IBD. This is because loperamide is available without a prescription and diarrhea is usually one of the first symptoms of IBD, which may take considerable time to accurately diagnose (Hendrickson et al., 2002).

Another major drawback of oral loperamide, the drug most commonly used for diarrhea in IBD, is that it can require several hours to take full effect. When used for the treatment of acute diarrhea, oral loperamide requires 9-20 hours to achieve symptomatic control (Van den Eynden et al., 1995; Kaplan et al., 1999). Patients who are not aware of this long lag period may prematurely take additional loperamide doses after the initial dose fails to control diarrheal symptoms after a “reasonable” period of time. Excessive use of loperamide in these patients greatly increases the risk of adverse effects such as abdominal pain, cramping, tenesmus, nausea, and bloating (Tuteja et al., 2004; Scarlett 2004).

Evidence from animal studies suggest that use of opioid antidiarrheals in IBD aggravates intestinal inflammation, leading to an overall increase in diarrheal symptoms. Morphine is known to enhance bacterial overgrowth and increase the rate of bacterial translocation, both of which increase inflammation (Kueppers et al., 1993; Runkel et al., 1993; Nieuwenhuijs et al., 1999; Yigitler et al., 2004). Bacterial translocation is the passage of intestinal microorganisms across the intestinal barrier to the mesenteric lymph nodes, liver and then to the systemic circulation, potentially resulting in sepsis, multi-organ failure, and shock (Duffy 2000; Lichtman 2001; Cevikel et al., 2003; Kruis 2004).

Increased bacterial translocation is primarily due to the antimotility effect of morphine, which increases intestinal transit times (Erbil et al., 1998; Sartor 2004; Wood et al., 2004). When morphine is administered simultaneously with tumor necrosis factor in rats, intestinal permeability is increased and bacterial translocation is enhanced to an even greater degree (Leslie et al., 1994). Tumor necrosis factor levels are known to be elevated in IBD patients (Farrell et al., 2002; Poullis et al., 2002; Su et al., 2002; Brunton et al., 2005). Since diphenoxylate, difenoxin, and loperamide all have greater antimotility effects than morphine, they would also be expected to exacerbate intestinal inflammation by increasing bacterial overgrowth and translocation.

Use of antidiarrheal agents is contraindicated in patients with moderate to severe colonic inflammation because they can precipitate the development of toxic megacolon. Toxic megacolon is a severe, potentially fatal complication of colitis that often requires immediate surgical intervention. It is characterized by segmental or total dilatation of the colon, abdominal distention, fever, septic shock, or perforation of the colon. Toxic megacolon is mainly seen in patients with ulcerative colitis or Crohn's colitis (Brown 1979; Fine et al., 1999; Salazar-Lindo et al., 2000; Hendrickson et al., 2002; Carter et al., 2004; Kornbluth et al., 2004). Excessive use or overdose of opioid antidiarrheals or any agents that slow colonic motility is strongly associated with increased risk of toxic megacolon in patients with severe colitis (Gan et al., 2003; Scarlett 2004; Brunton et al., 2005). Opioid antidiarrheals are also associated with colon ischemia, which itself can lead to the development of toxic megacolon (Walker et al., 2004). The severity and uncontrollability of diarrhea in IBD is in direct proportion to the severity of the intestinal inflammation. This effectively means patients with moderate to severe IBD who are most in need of symptomatic diarrheal control have no safe therapeutic options available to them.

The major limitations of opioid antidiarrheal drugs as they are currently used for treatment of chronic diarrhea, particularly in IBD are first, that they are contraindicated in patients with severe colonic inflammation, and second, that they are not suitable for continuous or long term use. Suppression of intestinal motility for long period of time by antidiarrheal opioids is the primary cause of their most serious gastrointestinal side effects such as megacolon and exacerbation of inflammation, particularly, when used in IBD.

Therefore, methods for management of long term diarrhea, particularly for IBD are needed.

It is further known that mast cell hyperplasia and degranulation are involved in the pathogenesis of several gastrointestinal diseases that present with diarrheal symptoms, including idiopathic inflammatory bowel disease, chronic ulcerative colitis, Crohn's disease, gastritis, collagenous colitis, irritable bowel syndrome, and chronic inflammatory duodenal bowel disorders (He et al., 2004; Nishida et al., 2002; Stoyanova et al., 2002; O'Sullivan et al., 2000; Gelbmann et al., 1999; Schwab et al., 1998; Beil et al., 1997; Bischoff et al., 1996; Fox et al., 1993). It is thought that the release of proinflammatory mediators such as histamine by mast cells in these diseases stimulates gastrointestinal motility and increases secretion, contributing to symptoms of diarrhea and abdominal pain (Park et al., 2006).

Mast cells are present within all layers of the gut wall throughout the entire gastrointestinal tract. They are an important source of several potent inflammatory mediators including histamine, neutral proteinases, proteoglycans, prostaglandins, leukotrienes, and certain cytokines. Due to the large number of biologically active substances mast cells release, they have profound effects on gut function, including fluid and electrolyte transport, particularly in disease states (Crowe et al., 1997).

Mast cells have a high capacity to release both preformed and newly-synthesized inflammatory mediators in response to environmental stimuli, such as allergen exposure. Crosslinking of IgE bound to high-affinity receptors on the surface of mast cells causes degranulation and rapid release of the preformed mediators histamine and neutral proteinases, in addition to sustained de novo synthesis and release of cytokines, chemokines and growth factors. Several other substances are known to activate or potentiate mediator release from mast cells, including certain opioids, neuropeptides, highly basic compounds, bee venom peptides, and adenosine (Xie et al., 2005; Barrett 2004; Bischoff et al., 1999). Recent studies of human colonic mast cells have shown that histamine and proteases, including trypsin and mast cell tryptase itself, can also trigger mast cell activation and degranulation (He et al., 2004a, 2004b, 2004c).

In Crohn's disease the rate of jejunal histamine secretion is elevated compared to healthy controls, and the secretion of histamine is correlated with disease activity, indicating that mast cell degranulation is involved in Crohn's pathogenesis (Knutson et al., 1990). Greatly increased gastrointestinal histamine levels are also found in ulcerative colitis and allergic enteropathy. Patients with active Crohn's disease or ulcerative colitis have increased levels of urinary N-methyl-histamine, a stable metabolite of mast cell-derived histamine, while enhancement of histamine metabolism is also observed in collagenous colitis and food allergy (Xie et al., 2006; Schwab et al., 2003; Winterkamp et al., 2002; Weidenhiller et al., 2000; Raithel et al., 1995). Increased rates of mast cell tryptase secretion are found both in non-inflamed and inflamed tissue of ulcerative colitis (Park et al., 2006). Mast cells isolated from resected colons of patients with active Crohn's disease or ulcerative colitis are able to release more histamine than mast cells from normal colon (Fox et al., 1993). Histamine concentration are elevated though the entire bowel wall of the colon and ileum of Crohn's disease patients as compared with controls, and mast cell numbers are markedly increased in Crohn's strictured bowel segments (Gelbmann et al., 1999).

One severe side effect of opioid treatment is that certain opioids have long been known to trigger the activation and degranulation of mast cells, resulting in the release of histamine and the serine proteinase tryptase. Symptoms suggestive of histamine release, such as wheal and flare around the injection site, peripheral vasodilatation, and bronchoconstriction are commonly reported after administration of intravenous opioids (Rook et al 2006; Grossmann et al., 1996; Schug et al., 1992). Elevated plasma histamine due to opioids is associated with several adverse effects such as hypotension, urticaria, pruritus, tachycardia, edema and hemodynamic instability. (Gordon et al., 2004; Barke et al., 1993; Ebertz et al., 1986). The opioids morphine, codeine, meperidine, and diacetylmorphine (heroin) are potent mast cell degranulators, whereas fentanyl and its congeners alfentanil, sufentanil, and remifentanil produce only very slight activation of mast cells (Rook et al., 2006; Brunton et al., 2005; Blunk et al., 2004).

Administration of opioids is known to produce nausea and vomiting in a large fraction of patients, an effect produced by the direct stimulation of the chemoreceptor trigger zone for emesis in the area postrema of the medulla (Brunton et al., 2005). Opioid-associated nausea and vomiting is particularly prevalent in the post-operative setting. Some investigators have suggested that histamine released from mast cell is at least partially responsible for post-operative nausea (Doenicke et al., 2004, 1994).

As compared to morphine and codeine, the synthetic opioids loperamide, diphenoxylate, difenoxin are all highly potent antidiarrheal agents. Difenoxin is seventy-seven times as potent as codeine, loperamide forty times as potent, and diphenoxylate fifteen times as potent (Awouters et al., 1983; Brunton et al., 2005). The antidiarrheal action of difenoxin is also significantly faster and more predictable than that of diphenoxylate (Rubens et al., 1972). Loperamide is currently considered the antidiarrheal agent of choice due to its low toxicity and because it has virtually no potential for narcotic abuse (Schinkel et al., 1996; Palmer et al., 1980; Niemegeers et al., 1979; Pelemans et al., 1976; Brunton et al., 2005).

Despite their high potency, loperamide, diphenoxylate, and difenoxin (as well as morphine/codeine) are surprisingly inadequate for treatment of chronic diarrhea due to inflammatory bowel disease, particularly in long-term or continuous use. Daily dosing with these agents tends to produce severe side effects such as tenesmus, nausea, cramping, abdominal pain, and bloating (Kornbluth et al., 2004; Rao 2004; Tuteja et al., 2004; Scarlett 2004). A recent survey of 307 Crohn's disease patients by Zutchi et al (2006) revealed that antidiarrheals ranked first among medications that are most in need of improvement.

Evidence from animal studies suggest that the use of opioid antidiarrheals in inflammatory bowel disease tends to aggravate intestinal inflammation. This is thought to be due to the antimotility effects of mu-opioid receptor agonists, which lengthen intestinal transit time. This may lead to bacterial overgrowth and increased bacterial translocation across the gastrointestinal mucosal barrier, potentially resulting in sepsis, multi-organ failure, and shock (Sartor et al., 2004; Wood et al., 2004; Yigitler et al., 2004; Duffy 2000; Erbil et al., 1998). In patients with severe colonic inflammation, the overuse of opioid antidiarrheals is associated with the development of toxic megacolon, a potentially fatal complication of colitis (Carter et al., 2004; Kornbluth et al., 2004; Fine et al., 1999).

Although they are widely used for diarrheal control due in inflammatory and allergic gastrointestinal conditions, the propensity of loperamide, diphenoxylate, and difenoxin to trigger mast cell degranulation has not been studied. There has been one reported case of anaphylaxis due to loperamide in a child (Perez-Calderon et al., 2003). Rash, pruritus, urticaria, angioedema, and swelling of the gums have all been reported as adverse events occurring after oral administration of loperamide or diphenoxylate (Janssen Pharmaceutica Inc., Imodium prescribing information, 1998; G.D. Searle & Co., Lomotil prescribing information, 2001). These adverse reactions are consistent with the effects of histamine release, therefore loperamide, diphenoxylate, or difenoxin may provoke mast cell degranulation under certain conditions.

Intestinal mast cell activation-degranulation after administration of opioid antidiarrheals may explain why these drugs are inadequate for symptomatic diarrheal control in many patients with inflammatory bowel disease. Mediators released from intestinal mast cells would increase secretion and gut motility, aggravate mucosal inflammation, and produce abdominal pain, cramping, tenesmus and nausea. Long-term use of mast cell degranulating agents in IBD patients could easily produce clinically significant exacerbations of disease activity.

Given the prominent role of mast cell hyperplasia and degranulation in the pathogenesis of these disorders, there exists a great need for effective antidiarrheal treatments that do not promote mast cell activation.

### SUMMARY OF THE INVENTION

In one embodiment, the present invention is directed to methods of treating and/or managing diarrhea, particularly management of long term diarrhea, such as diarrhea associated with inflammatory bowel disease (IBD). The method is based on using sequential effect of opioid agonists and antagonists.

In this diarrhea treatment and/or management methods of the invention, the emphasis is not cure but rather, management of the uncontrolled bowel movements. Accordingly the terms management and treatment are used a synonymous throughout the specification. These refer to methods that allow the individual suffering from diarrhea to manage the symptoms, i.e. the bowel movements so that there can be periods of no bowel movements during the day and when the time is more convenient for bowel movements, the method allows reversal of the blocking of the bowel movements. As such, the method allows an individual some time of normalcy, i.e. time when the bowel movement is controlled, during day or night.

In one embodiment, the invention provides a method of treating and/or managing diarrhea by administering to an individual in need thereof, a first composition, in a pharmaceutically acceptable carrier, comprising an effective amount of opioid agonist to suppress gut motility and secretion, and after a desired time interval, administering to the individual a second composition, in a pharmaceutically acceptable carrier, comprising an opioid receptor antagonist in sufficient amount to reverse the effects of the opioid agonist.

In one embodiment, the individual has a chronic diarrhea.

In another embodiment, the individual has IBD.

The time interval may be about 2, 3, 4, 5, 6, 7, 8, 9, 10, 11, 12, 13, 14, 15, or about 16 hours. One should preferably have at least about 8 hours of full baseline gastrointestinal motility within any 24 hour period. Generally, the more severe the inflammation in the gut, the shorter the blockage time should be. This can be experimentally determined by beginning with a shorter period of time and then gradually increasing gut motility suppression time. If the patient experiences pain towards the end of the suppression period, the time interval should be shortened to a maximum of a substantially painless period of time.

In one embodiment, the opioid receptor agonist and antagonist is directed to a mu-receptor (μ-receptor).

In one embodiment, Loperamide (for example 12 mg) is administered orally in a single dose, preferably in a liquid formulation. After 8 hours, a single dose of oral naltrexone (8 mg) is administered to reverse the effects of the loperamide. The duration of the antagonist action of naltrexone is likely to be at least 24 hours in this embodiment, therefore daily dosing with loperamide is not possible. This combination of oral loperamide/oral naltrexone is suited for patients with a high degree of colonic inflammation.

In another embodiment, a single dose of loperamide sufficient to provide antidiarrheal control for example 8 mg is administered by intravenous, intramuscular, or subcutaneous injection. Parenteral administration of loperamide provides a much more rapid and reliable onset of antidiarrheal action than does oral administration. Patients may self-administer the injection using a prefilled device such as an autoinjector, pen injector, or syrette. After 10 hours, a single dose of oral naltrexone (e.g., 10 mg) is administered to reverse the effects of the loperamide. This embodiment is suitable for use in patients who are not chronically dependent on antidiarrheal drugs, but still experience transient attacks of severe diarrhea requiring rapid control.

In another embodiment, a single-tablet formulation containing both an antidiarrheal mu-opioid receptor agonist in addition to a opioid antagonist is created as follows: 1) the agonist is contained within a rapidly dissolving excipient, and 2) the antagonist is contained within a delayed-release matrix so that none of the antagonist will be released until 6 hours after ingestion. The tablet is formulated to contain for example 2 mg of the agonist difenoxin and for example 4 mg of the antagonist nalmefene. This embodiment is useful in patients for whom dosing regimen compliance is an issue.

In another embodiment, oral diphenoxylate is administered (17.5 mg/day) in single or divided doses for diarrheal control over an extended period of seven days. At a specific time each day, a single injection of naloxone (3 mg) is administered so as to provide total reversal of the antidiarrheal effect for at approximately 3 hours. Since diphenoxylate has a much longer duration of action than naloxone, the antidiarrheal effect will return once the naloxone has been metabolized and eliminated. A second injection of naloxone (3 mg) is administered 8 hours after the first injection. The injections of naloxone are repeated each day the diphenoxylate is administered. This embodiment is suitable for patients who have, for example, diarrhea that is referred to as continuous and relatively uncontrollable. Such patients suffer, for example, from fecal incontinence because they cannot control the timing of the diarrhea. Such diarrhea is often experienced by patients suffering from IBD, IBS, and Crohn's disease.

In another embodiment, a transdermal patch containing loperamide is formulated so that the wearer receives a continuous dose of 6 mg/day over an extended period of seven days. At a specific time each day, a single injection of methylnaltrexone (0.5 mg/kg) is administered to reverse the effects of the loperamide for at least 10 hours. Since loperamide has a longer duration of action than methylnaltrexone, the antidiarrheal effect will return once the methylnaltrexone has been metabolized and eliminated. This embodiment is suitable for use in patients with “continuous” diarrhea who are taking opioid analgesics or patients with small-bowel inflammation in whom the absorption of oral drugs is compromised.

In another embodiment, a single dose of loperamide (4 mg) is administered via intranasal spray. Repeated loperamide doses are administered over a period of one hour, with 20 minutes between each dose, until diarrhea is controlled. After 8 hours, a single dose of oral alvimopan (4 mg) is used to reverse the effects of the loperamide. Due to the long duration of action of alvimopan, daily dosing with loperamide in this embodiment is not possible. This embodiment is suitable for patients who are concurrently taking opioid analgesics for pain control.

In yet another embodiment, at a specific time each day, a single dose of oral difenoxin (2 mg) is administered to effect control of diarrhea. Exactly 9 hours after the difenoxin dose, a single dose of methylnaltrexone (0.5 mg/kg) is administered by intranasal spray to reverse the effect of the difenoxin. This dosing regimen is repeated once per day, for five consecutive days. The short 3 hour elimination half-life of methylnaltrexone allows for daily dosing with difenoxin, since 15 hours (five half-lives) should be sufficient to completely eliminate methylnaltrexone from the systemic circulation. This embodiment is suitable for patients with mild inflammation who have fecal incontinence.

In still another embodiment, an extended-release injectable formulation of diphenoxylate that delivers a continuous dose of 7.5 mg/day over a period of ten days is administered. At a specific time each day, naloxone (3 mg) is administered via intranasal spray. Four hours after the first naloxone dose, a second 4 mg dose of naloxone is administered via intranasal spray. This naloxone dosing regimen is repeated once per day for ten days. The extremely short half-life of naloxone, approximately 60 minutes, necessitates two consecutive doses to reverse the effects of diphenoxylate for 8 hours. This embodiment is particularly suitable for patients that have “continuous” diarrhea.

In another embodiment, an inhaled nebulized formulation of loperamide (6 mg) is administered to achieve diarrheal control. After 10 hours, a transdermal cream containing nalmefene (4 mg in 2 ml cream base) is applied to the skin to reverse the effect of the loperamide. Two hours after the effects of loperamide have been reversed, the nalmefene cream is washed off. Due to the long duration of action of nalmefene, daily dosing with this regimen is not possible. This embodiment is suitable for patients receiving total parenteral nutrition.

In another embodiment, a solid oral transmucosal matrix formulation of diphenoxylate (15 mg) on a handle, designed to release 2.5 mg/hour is administered by being placed between the cheek and gum. Once satisfactory diarrheal control has been achieved, the diphenoxylate matrix is removed from the patient's mouth. After 10 hours, oral methylnaltrexone (8 mg/kg) is administered to reverse the effect of the difenoxin. This embodiment is suitable for use in patients who require occasional control of diarrheal symptoms.

In still another embodiment, oral codeine phosphate (30 mg) is administered to control diarrhea. After 4 hours, oral naloxone (15 mg) is administered to reverse the gastrointestinal effects of the codeine. Since both codeine and naloxone have short elimination half-lives, long-term once-daily dosing with this regimen is possible. This embodiment is suitable for patients with severe diarrhea with a high degree of inflammation.

In another embodiment, a transdermal cream containing loperamide (12 mg in 2.5 ml cream base) is applied to the skin to control diarrhea. After 8 hours, a transmucosal pharmaceutical chewing gum containing naloxone (10 mg), designed to release 0.75 mg/hour is alternately chewed and then held between the cheek and gingiva. The gum is chewed for 4 hours and then discarded, reversing the effect of the loperamide for approximately 6-7 hours. Due to the long duration of action of loperamide, the antidiarrheal effect will return once the naloxone has been eliminated. This embodiment is particularly suitable for patients that have difficulty absorbing oral drugs due to impaired intestinal function.

This invention further provides methods of controlling diarrhea using the opioid agonist and antagonist regime and simultaneously controlling, inhibiting or preventing intestinal mast cell activation/degranulation.

One can also use the method of controlling inhibiting or preventing intestinal mast cell activation/degranulation in combination with one antidiarrheal compound or a combination of antidiarrheal compounds as described in this specification.

Accordingly, in one embodiment the method comprises administering to an individual in need thereof, a formulation comprised of at least two pharmaceutical agents that is administered to a patient to control symptoms of diarrhea. The first agent is always an antidiarrheal. The second, third, fourth, or additional agents are selected from the group consisting of: mast cell stabilizers, histamine H1, H2, H3, and H4 receptor antagonists/antihistamines, inhibitors or substrates of cytochrome P450 isoform 2D6 (CYP2D6), antinauseants, antiemetic agents, tryptase inhibitors, and other antidiarrheal agents. The agents may be administered simultaneously or individually at different times, or any in combination thereof. As utilized in ACIID, these additional agents are intended to suppress mast cell activation and degranulation, attenuate the effects of mast cell-derived histamine and tryptase, reduce the severity of histamine- and opioid-associated nausea, and when the antidiarrheal agent is diphenoxylate or difenoxin, slow the metabolic transformation of the antidiarrheal agent by CYP2D6.

The antidiarrheal compositions for irritable or inflammatory disorders (ACIID) also encompasses a method for administering antidiarrheal treatment to a patient over a period of two or more consecutive days. This method is termed Consecutive Day Dosing with ACIID (CDDA). The general embodiment of CDDA is described as follows: On the first day of treatment, an ACIID formulation containing one or more opioid antidiarrheal agents is administered. On the second day of treatment the ACIID formulation is adjusted so that it contains reduced amount(s) or none of the opioid antidiarrheal agent(s), and increased, unchanged or additional amounts of any histamine receptor antagonists, mast cell stabilizers, antinauseants, antiemetic agents, or tryptase inhibitors. If the ACIID formulation is administered on the third or successive days, it is further adjusted so that it always contains a smaller dose of opioids than the formulation administered on the preceding day, or it may not contain any opioid antidiarrheal agents at all. The objective of treatment using CDDA is to provide the patient with adequate antidiarrheal control for more than one day while minimizing the use of opioids.

In one embodiment of CDDA, the long-acting synthetic opioids loperamide, diphenoxylate, or difenoxin, alone or in combination, are used in a formulation that includes histamine H1 receptor antagonists, histamine H2 receptor antagonists, mast cell stabilizers, and antinauseants. When difenoxin or diphenoxylate are used, their duration of action may be increased by the addition of inhibitors or substrates of CYP2D6. Alternatively, the short-acting antidiarrheal opioids codeine or morphine may be utilized in CDDA if they delivered using extended- or sustained-release formulations, or if they are used in combination with a longer-acting antidiarrheal agent.

In another embodiment, ACIID or CDDA may be used in combination with the Duration-Restricted Antidiarrheal Effect (DRAE) disclosed in U.S. Provisional Patent Application Ser. No. 60/784,661 (Siddiqi, 2006). DRAE is a method for the management of chronic diarrhea that utilizes mu-opioid receptor agonist antidiarrheal agents in combination with opioid antagonists. DRAE limits certain side effects of opioid antidiarrheals by reversing their antimotility and antisecretory effects after a predetermined length of time has elapsed, thereby returning gastrointestinal activity to baseline levels. Since DRAE does not prevent opioid-induced mast cell degranulation and histamine release, it can easily be combined with ACIID or CDDA to provide patients with an even greater reduction of the adverse effects caused by antidiarrheal treatment.

In yet another embodiment of ACIID or CDDA used with DRAE, an ACIID formulation containing one or more opioid antidiarrheals in combination with histamine receptor antagonists, mast cell stabilizers and antinauseants is used to control diarrhea. After a predetermined length of time has elapsed, generally 4 to 24 hours, for example 4, 5, 6, 7, 8, 9, 10, 11, 12, 13, 14, 15, 16, 17, 18, 19, 20, 21, 22, 23, or 24 hours, one or more opioid antagonists are administered to selectively reverse only the effects of the antidiarrheal opioid or a combination of opioids. This regimen may be repeated over the course of several days.

In one embodiment of ACIID is a formulation comprised of a peripheral mu-opioid receptor agonist antidiarrheal agent in combination with a histamine H1 receptor antagonist that also possesses antinauseant activity.

In another embodiment of ACIID is a formulation comprised of a peripheral mu-opioid receptor agonist antidiarrheal agent in combination with a histamine H1 receptor antagonist that has mast-cell stabilizing properties.

In yet another embodiment of ACIID is a formulation comprised of diphenoxylate or difenoxin in sufficient amount to produce an antidiarrheal effect in combination with an inhibitor of cytochrome P450 isoform 2D6 (CYP2D6) that is also a histamine H1 receptor antagonist.

In another embodiment of ACIID is a formulation comprised of diphenoxylate or difenoxin in sufficient amount to produce an antidiarrheal effect in combination with a substrate of CYP2D6, along with a histamine H1 receptor antagonist.

In another embodiment of ACIID is a formulation comprised of diphenoxylate or difenoxin in sufficient amount to produce an antidiarrheal effect in combination with an opioid substrate of CYP2D6, along with a non-sedating histamine H1 receptor antagonist, and a histamine H2 receptor antagonist.

In another embodiment of ACIID is a formulation comprised of a systemic mu-opioid receptor agonist antidiarrheal agent in combination with a non-sedating histamine H1 receptor antagonist.

In another embodiment of ACIID is a formulation comprised of a peripheral mu-opioid receptor agonist antidiarrheal agent in combination with a non-sedating H1 antihistamine and a histamine H2 receptor antagonist.

In another embodiment of ACIID is a formulation comprised of two different peripheral mu-opioid receptor agonist antidiarrheal agents in sufficient amounts to produce an antidiarrheal effect in combination with a histamine H1 receptor antagonist, a histamine H2 receptor antagonist, and an antinauseant.

### DETAILED DESCRIPTION OF THE INVENTION

The present invention is directed to a management method for diarrhea, wherein sequential administration of opioid agonist allows one to suppress gut motility and therefore block bowel movements for a determined time interval, and a sequentially administered opioid antagonist returns the mobility to the base level. The method allows one to manage time intervals for bowel movements. The method also allows a long term use of diarrhea control by opioid receptor agonists. The method further allows one to use opioid receptor agonists in even patients with severe IBS.

### Pharmacologic Therapy for Diarrhea

The rationale underlying the treatment of diarrhea is to lower the frequency, water content, and volume of stools by reduction of intestinal motility and secretion (Manatsathit et al., 2002). Opioids are the most widely used agents for symptomatic control of diarrhea. Specific opioid drugs commonly used as antidiarrheal agents include morphine, codeine, difenoxin, diphenoxylate, and loperamide (De Luca et al., 1996; Fine et al., 1999).

Opioids have intestinal antimotility, antisecretory, and proabsorptive effects which are produced by agonist activity at opioid receptors within the gastrointestinal tract (Manatsathit et al., 2002). The gastrointestinal effects of opioids are mediated primarily via peripheral mu-opioid receptors (Camilleri et al., 2004; Brunton et al., 2005). Mu-opioid receptors in the central nervous system (CNS) mediate the other well-known effects of opioid agonists, including analgesia, sedation, respiratory depression, and dependence (De Schepper et al., 2004). Clinically, antidiarrheal opioids lengthen intestinal transit time, decrease bowel movement frequency, reduce fecal volume, and lessen fluid and electrolyte loss (Killinger et al., 1979; Wood et al., 2004).

The opioids morphine and codeine are widely used as analgesics. Oral forms of morphine for control of diarrhea include deodorized tincture of opium and paregoric (camphorated opium tincture), whereas codeine is commonly administered as codeine phosphate. Codeine itself has very low affinity for opioid receptors. The antidiarrheal and analgesic effects of codeine are in fact due to its partial conversion into morphine via O demethylation by CYP2D6 (Mikus et al., 1997; De Schepper et al., 2004).

Morphine and codeine are rapidly-acting agents, reaching peak plasma concentration 30-90 minutes after oral administration, and have elimination half-lives (t½) of about 2 and 3 hours, respectively. These relatively short half-lives necessitate repeated dosing for adequate symptomatic control of diarrhea (Brunton et al., 2005). When used as antidiarrheal agents, both morphine and codeine have very high potential for abuse and addiction (Wingate et al., 2001; Greenwood-Van Meerveld et al., 2004; Rao 2004; Tuteja et al., 2004).

The synthetic opioids difenoxin and diphenoxylate are mu-receptor agonists that weakly penetrate the CNS, and as such, they have lower abuse/addiction potential than morphine. After oral administration, diphenoxylate is de-esterified to difenoxin (diphenoxylic acid), and reaches peak plasma levels in approximately 2 hours (Karim et al., 1972). The elimination half-life of difenoxin is 7-12 hours (Jackson et al., 1987; Brunton et al., 2005). Diphenoxylate is somewhat more potent than morphine in its antidiarrheal effects, while difenoxin is five times as potent as diphenoxylate (Awouters et al., 1983).

The apparent half-life of difenoxin produced by de-esterification of diphenoxylate is reported to be approximately 50 minutes in healthy volunteers (Jackson et al., 1987). This half-life may be longer in individuals who are ill or are concurrently taking other medications. A lower rate of diphenoxylate conversion to difenoxin appears likely, particularly in children, since delayed opioid toxicity has been reported to occur more than 24 hours after ingestion of diphenoxylate (Rumack et al., 1974; Wasserman et al., 1975; Block et al., 1977; Curtis et al., 1979; McCarron et al., 1991).

Diphenoxylate/difenoxin can produce CNS effects such as euphoria and respiratory depression at dosage levels about three times higher than the maximum recommended daily dose for diarrheal control. The CNS effects of diphenoxylate are substantial enough to allow its therapeutic use for reduction of withdrawal symptoms during detoxification of narcotic-addicted individuals (Glatt 1972; Ives et al., 1983; Kleinman et al. 1997; Jimenez-Lerma et al., 2002). A subtherapeutic amount of the anticholinergic atropine sulfate is added to commercially available preparations of diphenoxylate or difenoxin. The addition of atropine is intended to increase the unpleasant side effects of large doses of diphenoxylate or difenoxin, thereby discouraging deliberate overdosage and lowering abuse potential (Brunton et al., 2005).

Loperamide, a peripheral mu-receptor agonist, is a piperidine butyramide derivative that is 40-50 times more potent than morphine as an antidiarrheal agent (Salazar-Lindo et al., 2000; Brunton et al., 2005). At therapeutic oral doses loperamide has essentially no CNS effects at all, and virtually no abuse potential (Jaffe et al., 1980). Loperamide is efficiently removed from the CNS because it is an excellent substrate for the P-glycoprotein efflux pump in the blood-brain barrier (Schinkel et al., 1996; Kharasch et al., 2004). P glycoprotein is the product of the multiple drug resistance gene MDR1. In MDR knockout mice, CNS concentrations of loperamide are eight times higher than in wild-type mice, and striking, potentially lethal opioid CNS effects such as respiratory depression are observed. When the P-glycoprotein inhibitor quinidine is co-administered with loperamide in humans, pharmacodynamic respiratory changes are produced, signifying increased loperamide levels within the CNS (Schinkel et al., 1996; Sadeque et al., 2000; Wandel et al., 2002). Loperamide has several other effects that are not mediated by opioid receptors, including calcium channel blockade and functional inhibition of calmodulin (Harper et al., 1997; Daly et al., 2000).

Loperamide reaches peak plasma levels 3-5 hours after oral administration, with an elimination half life of 9-14 hours (Heel et al., 1978; Mcguire et al., 1978; Jaffe et al., 1980; Ericsson et al., 1990). Approximately 70% of a loperamide dose is absorbed in the gastrointestinal tract, but its systemic bioavailability is low due to enterohepatic cycling, high first pass metabolism, and fecal excretion (De Luca et al., 1996; Wingate et al., 2001; Yu et al., 2004). As compared to diphenoxylate/difenoxin, loperamide is a significantly more potent antidiarrheal agent, has a substantially longer duration of effect, and has a much more favorable safety profile (Pelemans et al., 1976; Niemegeers et al., 1979). For these reasons, loperamide is currently considered the antidiarrheal agent of choice and has been approved for over-the-counter distribution (Palmer et al., 1980; Schinkel et al., 1996; Brunton et al., 2005).

Opioid Bowel Dysfunction and its Treatment with Opioid Antagonists

Virtually all opioid analgesics used for primary pain control are mu-receptor agonists (Brunton et al., 2005). When opioid analgesics are chronically administered in patients with otherwise normal gastrointestinal function, a debilitating condition known as opioid bowel dysfunction (OBD) commonly results. OBD is characterized by chronic constipation, hard dry stools, incomplete evacuation, fecal impaction, bloating, nausea, vomiting, abdominal cramping, gastroesophageal reflux, pseudo-obstruction of the bowel, and inadequate absorption of oral drugs (Cheskin et al., 1995; Yuan et al., 2000; Hawkes et al., 2001; Schmidt 2001; Camilleri 2004; Grundy et al., 2004; Holzer 2004). Opioid bowel dysfunction has traditionally been managed by the use of laxatives and stool softeners, however, these treatments are ineffective in a large fraction of patients (Latasch et al., 1997; Foss 2001).

A newer approach to the management of OBD is to selectively antagonize opioid receptors in the gastrointestinal tract without affecting the opioid receptors in the CNS that mediate analgesia (Pappagallo 2001; Greenwood-Van Meerveld et al., 2004). The competitive opioid receptor antagonists naloxone, naltrexone, nalmefene, methylnaltrexone, and alvimopan have been studied for their efficacy in treating OBD (Gowan et al., 1988; Sykes 1996; Foss 2001; Holzer et al., 2004; Galligan et al., 2005) Naloxone, naltrexone, and nalmefene are excellent CNS penetrants, while methylnaltrexone and alvimopan do not cross the blood-brain barrier (Yuan et al., 2000; Schmidt 2001; Camilleri 2004).

Naloxone is a rapidly acting pure opioid antagonist that is approved for the treatment of opioid overdose. When administered via the intravenous or intramuscular routes, naloxone systemically reverses the effects of opioid-receptor agonists within one to two minutes. The elimination half life of naloxone is 30-90 minutes in healthy individuals (Clarke et al., 2002; Brunton et al., 2005). Naloxone is poorly absorbed following oral administration, but it can completely reverse the effects of opioid agonists within the gastrointestinal tract (Meissner et al., 2000; Camilleri 2004). Naloxone has been shown to reverse the antimotility and antisecretory effects of loperamide (Piercey et al., 1979; Caldara et al., 1981; Basilisco et al., 1985, 1987; Mellstrand 1987). The primary drawback of oral naloxone for the treatment of OBD is that it has a very narrow therapeutic index and tends to reverse analgesia and precipitate opioid withdrawal symptoms at doses only slightly higher than those needed to restore normal gut function. (Culpepper-Morgan et al., 1992; Cheskin et al., 1995; Sykes 1996; Latasch et al., 1997; Yuan et al., 2000; Hawkes et al., 2001; Holzer 2004)

Naltrexone is a pure opioid antagonist that undergoes nearly complete absorption from the gastrointestinal tract, reaching peak plasma levels about 1 hour after oral administration. Naltrexone is converted by extensive first-pass metabolism to 6-beta-naltrexol, which is also a pure opioid antagonist. The elimination half-life of naltrexone is 3-4 hours, while that of 6-beta-naltrexol is 13 hours (Brunton et al., 2005). Naltrexone is approximately 2.5 times more potent than naloxone and has a 20-fold greater affinity for mu-opioid receptors than morphine (Yeo et al., 2003). Naltrexone is known to cause dose-dependent hepatotoxicity when administered at high levels. Naltrexone does not display any selectivity toward opioid receptors in the gastrointestinal tract, so it is not useful for the treatment of OBD as it reverses analgesia (Yuan et al., 1999; Pappagallo 2001).

Nalmefene is a pure opioid antagonist that displays lower toxicity than naltrexone. After oral administration nalmefene is well absorbed from the gastrointestinal tract and has an elimination half-life of approximately 11 hours (Dixon et al., 1986; Gal et al., 1986; Frye et al., 1996). Nalmefene is considered to be a more potent agent than naloxone with a longer duration of clinically effective antagonist activity (Kim et al., 1997; Joshi et al., 1999; Brunton et al., 2005). Like naltrexone, nalmefene is nonselective in its opioid antagonist effects, and is not effective for the treatment of OBD (Cheskin et al., 1995; Yuan et al., 2000).

Methylnaltrexone is a N-methyl quaternary derivative of naltrexone that has low lipid solubility and does not cross the blood-brain barrier in humans. It does not reverse the CNS effects of opioid agonists and does not precipitate symptoms of opioid withdrawal (Yuan et al., 1996; Foss 2001). After intravenous administration, methylnaltrexone immediately reverses the reduction of gut motility and secretion produced by opioid agonists, without affecting analgesia. Methylnaltrexone has an elimination half life of 2-3 hours. When administered orally for OBD, methylnaltrexone restores normal bowel function within 4-7 hours (Yuan et al., 1997, 1998, 1999, 2000, 2002, 2005).

Alvimopan is a synthetic, peripherally acting, N-substituted piperidine derivative mu-opioid antagonist. Alvimopan does not penetrate the blood-brain barrier and is very poorly absorbed after oral administration (Camilleri 2004; Greenwood-Van Meerveld et al., 2004; Cassel et al., 2005; Delany et al., 2005; Galligan et al., 2005; Leslie et al., 2005). It is approximately 200 times more potent than methylnaltrexone in reversing the effects of morphine in the gastrointestinal tract, with a four-fold longer duration of antagonist activity. It is effective for the treatment of OBD (Schmidt 2001; Holzer 2004; Paulson et al., 2004; Camilleri 2005; Gonenne et al., 2005).

The present invention is directed to timed delivery of the opioid agonists and antagonists so that the typical problems, including tenesmus, rectal urgency, cramping, abdominal pain, and bloating, that are associated with opioid therapy, particularly with the long term opioid therapy, can be reduced or avoided.

Table 1 shows some opioid agonists and antagonists that can be used according to the present invention. Others can be readily substituted for these ones by one skilled in the art. The method of the present invention may be practiced with any opioid agonist/antagonist pair that are suitable for pharmaceutical use in humans.

TABLE 1

| Drug | Trade Name | Manufacturer | Status | Indication |
|---|---|---|---|---|
| Loperamide | IMODIUM ® | McNeil-PPC | off-patent | diarrhea |
| (oral) | | | | |
| Diphenoxylate | LOMOTIL ® | Searle | off-patent | diarrhea |
| (oral) | | | | |
| Difenoxin (oral) | MOTOFEN ® | Amarin | off-patent | diarrhea |
| Naltrexone (oral) | REVIA ® | DuPont Pharma | off-patent | alcohol |
| | | | | dependence |
| Naloxone | NARCAN ® | Endo | off-patent | opioid overdose |
| (injection) | | Pharmaceuticals | | |
| Nalmefene | REVEX ® | Baxter | U.S. Pat. No. | opioid overdose |
| (injection) | | | 4,535,157 | |
| Nalmefene (oral) | | BioTie | Phase II/III | nicotine |
| | | Therapies and | | dependence, |
| | | Somaxon | | impulse control |
| | | Pharmaceuticals, | | disorders |
| | | Inc., | | |
| Methylnaltrexone | | Progenies | Phase III | opioid bowel |
| (MNTX) | | Pharmaceuticals | | dysfunction |
| Alvimopan (oral) | ENTEREG ® | Adolor | Phase III | opioid bowel |
| | | | | dysfunction, |
| | | | | postoperative |
| | | | | ileus |
| Naltrexone | VIVITREX ® | Alkermes | Regulatory | alcohol |
| (long-acting | | | Review | dependence |
| injection) | | | | |
| Naltrexone (low- | PTI-901 | Pain | Failed Phase | irritable bowel |
| dose oral) | | Therapeutics | III (Dec 9, 2005) | syndrome |
| | | Inc. | | |
| Morphine (oral | AVINZA ® | Elan/Ligand | | severe pain |
| extended release) | | Pharmaceuticals | | |
| Morphine | DEPODUR ® | Endo | | pain |
| (extended release | | Pharmaceuticals | | |
| injection) | | | | |
| Fentanyl (oral | ACTIQ ® | Cephalon Inc. | | breakthrough |
| transmucosal) | | | | pain |
| Fentanyl | DURAGESIC ® | Janssen | | persistent pain |
| (transdermal) | | Pharmaceutica | | |

### The Duration-Restricted Antidiarrheal Effect (DRAE)

The present invention provides a new method for the management of chronic diarrhea and fecal incontinence in inflammatory bowel disease: the Duration-Restricted Antidiarrheal Effect (DRAE). The fundamental concept underlying DRAE is that the duration of the gastrointestinal effects of antidiarrheal therapy must be sharply restricted so that they only last a predetermined length of time, generally less than 18 hours, for example 8 hours, 10 hours, 12 hours. The clinical objective of treatment using DRAE is not to stop diarrhea, but instead to delay bowel movements to a more convenient time. Successful use of DRAE will provide a patient with a multiple-hour block of time in which no bowel movements occur, after which, the baseline gastrointestinal activity is rapidly restored. For the duration of the antidiarrheal effect, patients can attend work or school, or participate in social activities without having to remain close to toilet facilities.

The prompt restoration of gut motility using DRAE should greatly reduce or eliminate the adverse effects that are associated with chronic use of antidiarrheal agents, such as tenesmus, rectal urgency, nausea, cramping, abdominal pain, and bloating. Use of DRAE for management of chronic diarrhea in IBD would not be expected to reduce the total number of bowel movements or reduce the total stool volume—only the overall timing of the bowel movements is affected.

The exact duration of the antidiarrheal effect is determined by the severity of the inflammatory disease in the patient; in general, shorter durations are used for cases with higher degrees of inflammation. In patients with active disease who have severe inflammation, the antidiarrheal effect may safely last only 2-6 hours, while for patients with only mild inflammation, the appropriate duration may be 8-12 hours. Daily use of DRAE is possible as long as there is a minimum of 8 hours of full baseline gastrointestinal motility in any 24-hour period in which DRAE is administered. DRAE should allow safe, long-term antidiarrheal control even in IBD patients with high levels of colonic inflammation, in whom conventional antidiarrheal therapies are contraindicated.

DRAE is also useful for the management of chronic diarrhea and fecal incontinence due to other conditions such as irritable bowel syndrome (IBS), small-bowel bacterial overgrowth, short bowel syndrome, bile acid malabsorption, celiac disease, microscopic colitis, diabetic diarrhea, injury, or trauma.

In one embodiment, the diarrhea is “uncontrollable”, for example, when the individual has 5-40 or more bowel movements a day. With the exception of newborns, who can have stool 8-10 times a day, a child or an adult typically is experiencing diarrhea if they experience more than 4 bowel movements a day. Alternatively, the diarrhea is uncontrollable when the patient cannot predict the bowel movements and is suffering from fecal incontinence.

Typically, one is experiencing severe diarrhea if they experience a bowel movement more than 10 times a day, and specifically, if the bowel movement becomes unpredictable to and uncontrollable by the individual thus resulting in fecal incontinence.

### Delayed Pharmacologic Reversal of Antidiarrheal Activity (DPRAA)

In one embodiment of DRAE, the method comprises administering to an individual in need thereof, a formulation comprised of two active pharmaceutical agents. The first agent quickly suppresses gut motility and secretion, and the second agent rapidly reverses the effects of the first agent after a predetermined length of time has elapsed, thereby returning gut motility and secretion to baseline levels. This embodiment is termed Delayed Pharmacologic Reversal of Antidiarrheal Activity (DPRAA).

The preferred implementation of DPRAA is to use a mu-opioid receptor agonist to establish antidiarrheal control, and then, after a predetermined length of time, use a mu-opioid receptor antagonist to reverse the effects of the agonist. Complete reversal of the agonist effect is preferred, but partial reversal may be sufficient to allow for functional restoration of gut motility. The mu-receptor agonist may be peripheral or a CNS penetrant, however, peripheral mu-receptor agonists are preferred. Use of pure opioid antagonists as reversal agents is preferred over mixed agonist-antagonist or partial agonist opioids. The mu-opioid antagonist may be peripheral or a systemic CNS penetrant. In IBD patients that are not concurrently taking opioid analgesics, systemic opioid antagonists are preferred. In IBD patients that are concurrently taking opioid analgesics for pain control, peripheral opioid antagonists are preferred.

The opioid-receptor agonist and antagonist agents may be administered via several routes, including, but not limited to, oral, intravenous, intramuscular, subcutaneous, intralingual, sublingual, transmucosal, transdermal, intranasal, inhalation, intrarectal, or intravaginal routes. Formulations may be immediate-release, delayed-release, extended-release, or any combination thereof.

Antidiarrheal mu-opioid receptor agonists include, but are not limited to, morphine, codeine, difenoxin, diphenoxylate, and loperamide. Systemic opioid antagonists include, but are not limited to, naloxone, naltrexone, nalmefene, diprenorphine, naloxonazine, and naloxone benzoylhydrazone. Peripheral opioid antagonists include, but are not limited to, methylnaltrexone, alvimopan, N-methylnaloxone (naloxone methiodide), and N methylnalmefene.

Opioid antagonists useful in the methods of the present invention include, but are not limited to

Numerous formulations of opioids have been developed and studied, Examples of such formulations are described below. Others can be readily used in the diarrhea management method as described herein, and these examples should not be considered limiting.

Oral dosage forms have the advantage of simplicity and convenience, but suffer from inter-patient variability in the rate of drug absorption. This is a particular concern for patients who have impaired intestinal function as is commonly seen in IBD (Yuan et al., 1999). Oral dosing forms may be immediate-release, delayed-release, extended-release, or some combination thereof. Extended-release once-per-day oral formulations of morphine have been described (Eliot et al., 2002) as well as delayed-release enteric formulations of methylnaltrexone (Yuan et al., 2000).

Injection is the most rapid method of opioid drug delivery. However, not all patients are comfortable with injection and this route has the greatest potential for abuse and overdose. Intravenous, intramuscular, and subcutaneous administration of methylnaltrexone have been studied for treatment of opioid bowel dysfunction (Yuan et al., 1996, 2002, 2005). Intralingual injection of naloxone has been reported when the intravenous route is unobtainable (Maio et al., 1987). Extended-release injectable formulations of morphine (Gambling et al., 2005) as well as an ultra-long acting injectable formulation of naltrexone (Bartus et al., 2003; Dean 2005) have been described.

Transdermal administration of drugs slowly deliver a low continuous dose through the skin over a long period of time. Transdermal adhesive patches for the delivery of fentanyl (Portenoy et al., 1993), naltrexone (Hammell et al., 2004) and naloxone (Jaiswal et al., 1999; Panchagnula et al., 2001, 2005;) have been developed. A transdermal cream formulation of loperamide has been studied (Trottet et al., 2004). Transdermal iontophoretic systems for the delivery of analgesic opioids have also been described (Zempsky et al., 1998; Kalia et al., 2004; Ashburn et al., 1992, 1995).

Oral transmucosal delivery of drugs can be more rapid than oral administration, and tends to have greater bioavailability since first-pass metabolism in the liver is bypassed. Additionally it is more comfortable and convenient for patients than injection. Oral transmucosal fentanyl has been approved for the treatment of breakthrough cancer pain (Coluzzi et al., 2001; Zhang et al., 2002; Mystakidou et al., 2005). Sublingual morphine has also been studied for this purpose (Coluzzi 1998).

Inhalation of nebulized opioids is an alternative to injection for rapid administration. Inhalation of the opioid analgesics fentanyl and morphine has been studied for pain management (Chrubasik et al., 1988; Worsley et al., 1990; Higgins et al., 1991; Farncombe et al., 1994; Baydur 2004; Foral et al., 2004). The use of nebulized naloxone for the treatment of methadone intoxication has also been reported (Mycyk et al., 2003).

Intranasal spray is a another alternative to injection for rapid systemic drug delivery. The intranasal route has a rapid onset of effect, is painless, convenient, and is easily amenable to patient self-administration. Intranasal spray administration of opioid analgesics for pain management has been studied (Schwagmeier et al., 1995, 1996; Vachharajani, et al., 1996, 1997; Takala et al., 1997; Dale et al., 2002; Davis et al., 2004; Turker et al., 2004; Striebel et al., 1992, 1993, 1995, 1996). Intranasal naloxone has been used for the treatment of narcotic overdose (Barton et al., 2002, 2005; Kelly et al., 2002, 2005), in these cases, delivery of naloxone to the systemic circulation is comparable to that produced by intravenous injection.

Intrarectal formulations of opioid analgesics are available (Cole et al., 1990; De Conno et al., 1995; Warren 1996; Ripamonti et al., 1992, 1995, 1997;). This route may be used in patients who have difficulty swallowing or other oral pathology. As an alternative to the rectal route, intravaginal administration of morphine has also been reported (Benziger et al., 1983; Ostrop et al., 1998).

Another embodiment of DRAE is to administer a single antidiarrheal agent whose gastrointestinal effects are abolished completely within 18 hours after a single dose. This embodiment can be implemented using a Short-Acting Peripheral Mu-Opioid Receptor Agonist (SAPMORA) with an elimination half-life of less than four hours. In general, SAPMORAs with very short elimination half-lives (less than one hour) are preferred for use in patients with severe colonic inflammation. SAPMORAs with elimination half-lives in the range of 1-4 hours are preferred for patients with mild to moderate intestinal inflammation. Daily dosing with SAPMORAs is possible as long as there are at least 8 hours of full baseline gastrointestinal motility in any 24-hour period in which the SAPMORA is administered.

Available SAPMORAs include, but are not limited to, the ring nitrogen quaternary ammonium derivatives of short-acting opioid analgesics. Examples include, but are not limited to, N-methylmorphine, N-methylcodeine, N-methyldihydrocodeine, N-methylhydrocodone, N-methylhydromorphone, N-methyletorphine, N-methylfentanyl, N-methylsufentanil, N-methylalfentanil, and N-methylremifentanil. SAPMORAs with elimination half-lives in the range of 1-4 hours can be administered in a single dose to provide a few hours of antidiarrheal control. Successive doses may be administered if control is required for a longer period. SAPMORAs with elimination half-lives less of than 1 hour are preferably administered in a low continuous dose over the duration when antidiarrheal control is required, preferably via transdermal, transmucosal, or extended-release oral or injectable formulations.

Another embodiment of use of DRAE is to administer a formulation containing two active pharmaceutical agents that are administered simultaneously. The first agent is an antidiarrheal drug with an elimination half-life longer than four hours. The second agent is a drug that interacts with the first agent, reducing the elimination half-life of the first agent to less than four hours. Available antidiarrheal agents for use in this embodiment include, but are not limited to, difenoxin, diphenoxylate, and loperamide.

Naturally, the diarrheal management method of the present invention can be used in combination of treatment methods or methods to alleviate symptoms of IBD. IBD therapeutic agents that can be used in combination with the management method of the present invention include, but are not limited to, benzodiazepine compounds, antispasmodic, selective serotonin reuptake inhibitors (SSRIs), cholecystokinin (CCK) receptor antagonists, motilin receptor agonists or antagonists, natural killer (NK) receptor antagonists, corticotropin Releasing Factor (CRF) receptor agonists or antagonists, somatostatin receptor agonists, antacids, GI relaxants, anti-gas compounds, bismuth-containing preparations, pentosan polysulfate, anti-emetic dopamine D2 antagonists, prostaglandin E analogs, gonadotrophin-releasing hormone analogues (leuprolide), corticotrophin-1 antagonists, neurokinin 2 receptor antagonists, cholecystokinin-1 antagonists, beta-blockers, anti-esophageal reflux agents, anti-muscarinics, antidiarrheals, antiinflammatory agents, pro-motility agents, 5HT1 agonists, 5HT3 antagonists, 5HT4 antagonists, 5HT4 agonists, bile salt sequestering agents, bulk-forming agents, bulk-forming laxatives, cathartic laxatives, diphenylmethane laxatives, osmotic laxatives, saline laxatives, other laxatives, stool softeners, alpha-2-adrenergic agonists, mineral oils, antidepressants, herbal medicines, juices, fruits, vegetables, and herbal and vegetable juices. In another embodiment, the peripheral opioid antagonist is administered in a formulation comprising the peripheral opioid antagonist and an antibiotic.

### Management of Diarrhea and Reduction of Side Effects

Since both histamine and tryptase are each alone capable of triggering mast cell degranulation, He (2004) proposes that mast cells are capable of self-amplifying and maintaining their own activation through positive feedback. According to He's model, activation-degranulation of any given mast cell releases histamine and tryptase, which in turn activate nearby mast cells, increasing the histamine and tryptase levels even further in a paracrine positive feedback loop. Positive autocrine feedback is also possible in this model. Histamine has been suggested to act through H1 and H2 histamine receptors, while tryptase acts through its receptor PAR-2. This proposed mechanism at least partially explains the phenomenon of a single allergen contact event which provokes a local allergic response that lasts days or weeks in a sensitized individual (Xie et al., 2005; He et al., 2006, 2004).

In irritable bowel syndrome (IBS), mast cell numbers are increased in the gastrointestinal mucosa without the appearance of overt mucosal inflammation. Mast cell numbers are significantly increased in the mucosa of the terminal ileum, caecum, ascending colon and rectum of patients with IBS compared with controls (Park et al., 2006; O'Sullivan et al., 2000). Mast cells from the colonic mucosa of IBS patients are known to release increased amounts of the mediators histamine and tryptase, which in turn produce the gut sensory and motor dysfunction that is characteristic of IBS. The close proximity of these mucosal mast cells to enteric nerves fibers correlates well with the severity of abdominal pain perception in IBS (Barbara et al., 2006).

Opioid-induced activation and degranulation of mast cells is not mediated by immunoglobulins, nor is it mediated via the classical opioid receptors, and it is not a nonspecific effect of high opioid concentration (Rook et al 2006; Veien et al., 2000; Barke et al., 1993; Stellato et al., 1992). The pure opioid receptor antagonist naloxone does not induce histamine or tryptase release and does not attenuate mast cell activation when it is co-administered with morphine, codeine, or meperidine (Blunk et al., 2004; Doenicke et al., 1995; Hermens et al., 1985). Since opioid receptor binding does not appear to be involved, some investigators have suggested that these opioids directly interact with the G-proteins of mast cells, leading to degranulation (Blunk et al., 2004).

A large fraction of the volume of a mast cell is occupied by membrane-enclosed secretory granules. A single mature human mast cell contains 50 to 500 secretory granules each with a diameter ranging from 200 to 500 nm. The granules originate from the Golgi apparatus, which directs the synthesis and organization of the preformed mediators that they contain. The preformed mediators include histamine, a primary amine formed by decarboxylation of histidine, the neutral proteinases tryptase, chymase and carboxypeptidase, and the proteoglycans heparin and chondroitin sulfate. (Brunton et al., 2005; He 2004)

Diphenoxylate is a prodrug that is de-esterified to form the active metabolite difenoxin (diphenoxylic acid) by hepatic and intestinal esterases. In humans, difenoxin is primarily metabolized to form its glucuronic acid ester conjugate and p-hydroxydiphenoxylic acid (Karim et al., 1972). The aryl hydroxylation of difenoxin to form p-hydroxydiphenoxylic acid is most likely catalyzed by one or more cytochrome P450 isoforms, possibly including CYP2D6. Since diphenoxylate and difenoxin are both structurally related to the potent mast cell degranulator meperidine, it is probable that diphenoxylate and its metabolites are also mast cell degranulators.

Accordingly, this invention is also directed to a method for the symptomatic treatment of diarrhea, especially diarrhea due to diseases characterized by intestinal inflammation, mast cell hyperplasia, or mast cell degranulation. This method is broadly termed Antidiarrheal Compositions for Irritable or Inflammatory Disorders (ACIID).

The objective of treatment using ACIID is to provide the patient with adequate control of diarrheal symptoms while simultaneously reducing the severity of adverse effects that are caused by antidiarrheal agents. The adverse effects targeted for reduction using ACIID include, but are not limited to, mast cell activation and degranulation, the physiologic effects of elevated histamine levels, increased gastrointestinal mucosal secretion, nausea, vomiting, pruritus, urticaria, and angioedema. ACIID is primarily intended for those patients in whom conventional antidiarrheal treatments are inadequate or ineffective, particularly those suffering from inflammatory bowel disease or irritable bowel syndrome.

The general embodiment of ACIID is a formulation comprised of at least two pharmaceutical agents that is administered to a patient to control symptoms of diarrhea. The first agent is always an antidiarrheal. The second, third, fourth, or additional agents are selected from the group consisting of: mast cell stabilizers, histamine H1, H2, H3, and H4 receptor antagonists/antihistamines, inhibitors or substrates of cytochrome P450 isoform 2D6 (CYP2D6), antinauseants, antiemetic agents, tryptase inhibitors, and other antidiarrheal agents. The agents may be administered simultaneously or individually at different times, or any in combination thereof. As utilized in ACIID, these additional agents are intended to suppress mast cell activation and degranulation, attenuate the effects of mast cell-derived histamine and tryptase, reduce the severity of histamine- and opioid-associated nausea, and when the antidiarrheal agent is diphenoxylate or difenoxin, slow the metabolic transformation of the antidiarrheal agent by CYP2D6.

ACIID also encompasses a method for administering antidiarrheal treatment to a patient over a period of two or more consecutive days. This method is termed Consecutive Day Dosing with ACIID (CDDA). The general embodiment of CDDA is described as follows: On the first day of treatment, an ACIID formulation containing one or more opioid antidiarrheal agents is administered. On the second day of treatment the ACIID formulation is adjusted so that it contains reduced amount(s) or none of the opioid antidiarrheal agent(s), and increased, unchanged or additional amounts of any histamine receptor antagonists, mast cell stabilizers, antinauseants, antiemetic agents, or tryptase inhibitors. If the ACIID formulation is administered on the third or successive days, it is further adjusted so that it always contains a smaller dose of opioids than the formulation administered on the preceding day, or it may not contain any opioid antidiarrheal agents at all. The objective of treatment using CDDA is to provide the patient with adequate antidiarrheal control for more than one day while minimizing the use of opioids.

In a preferred embodiment of CDDA, the long-acting synthetic opioids loperamide, diphenoxylate, or difenoxin, alone or in combination, are used in a formulation that includes histamine H1 receptor antagonists, histamine H2 receptor antagonists, mast cell stabilizers, and antinauseants. When difenoxin or diphenoxylate are used, their duration of action may be increased by the addition of inhibitors or substrates of CYP2D6. Alternatively, the short-acting antidiarrheal opioids codeine or morphine may be utilized in CDDA if they delivered using extended- or sustained-release formulations, or if they are used in combination with a longer-acting antidiarrheal agent.

In another embodiment, ACIID or CDDA may be used in combination with the Duration-Restricted Antidiarrheal Effect (DRAE) disclosed in U.S. Provisional Patent Application Ser. No. 60/784,661 (Siddiqi, 2006). DRAE is a method for the management of chronic diarrhea that utilizes mu-opioid receptor agonist antidiarrheal agents in combination with opioid antagonists. DRAE limits certain side effects of opioid antidiarrheals by reversing their antimotility and antisecretory effects after a predetermined length of time has elapsed, thereby returning gastrointestinal activity to baseline levels. Since DRAE does not prevent opioid-induced mast cell degranulation and histamine release, it can easily be combined with ACIID or CDDA to provide patients with an even greater reduction of the adverse effects caused by antidiarrheal treatment.

In one preferred embodiment of ACIID or CDDA used with DRAE, an ACIID formulation containing one or more opioid antidiarrheals in combination with histamine receptor antagonists, mast cell stabilizers and antinauseants is used to control diarrhea. After a predetermined length of time has elapsed, generally 4 to 24 hours, one or more opioid antagonists are administered to selectively reverse only the effects of the antidiarrheal opioid(s). This regimen may be repeated over the course of several days.

The compositions of the present invention can also include a humectant to reduce or prevent drying of the mucus membrane and to prevent irritation thereof. Suitable humectants that can be used in the present invention include sorbitol, mineral oil, vegetable oil and glycerol; soothing agents; membrane conditioners; sweeteners; and combinations thereof. The concentration of the humectant in the present compositions will vary depending upon the agent selected.

Antidiarrheal agents suitable for use in ACIID include but are not limited to: opioid agonists such as morphine, codeine, loperamide, loperamide oxide, diphenoxylate, difenoxin, paregoric, tincture of opium, and nufenoxole; alpha-adrenergic receptor agonists such as clonidine, lofexidine, and lidamidine; enkephalinase inhibitors such as racecadotril (acetorphan); enkephalin analogs such as BW942C; somatostatin and its analogs such as octreotide; bile acid sequestrants such as cholestyramine, colestipol, and colesevelam; and other agents such as berberine.

Histamine H1 receptor antagonists include but are not limited to acrivastine, alimemazine, antazoline, astemizole, azatadine, azelastine, bamipine, bromazine, brompheniramine, buclizine, carbinoxamine, cetirizine, chlorcyclizine, chloropyramine, chlorphenamine, chlorpheniramine, chlorphenoxamine, cinnarizine, clemastine, cyclizine, cyproheptadine, deptropine, desloratadine, dexbrompheniramine, dexchlorpheniramine, dimenhydrinate, dimetindene, diphenhydramine, diphenylpyraline, dithiaden, doxepin, doxylamine, ebastine, emedastine, epinastine, fexofenadine, histapyrrodine, hydroxyethylpromethazine, hydroxyzine, isothipendyl, ketotifen, levocabastine, levocetirizine, loratadine, mebhydrolin, meclizine, meclozine, medizine, mepyramine, mequitazine, methapyrilene, methdilazine, mizolastine, mizolastine, olopatadine, oxatomide, oxomemazine, phenindamine, pheniramine, pimethixene, promethazine, pyrilamine, pyrrobutamine, rupatadine, talastine, terfenadine, thenalidine, tripelennamine, and triprolidine.

Histamine H2 receptor antagonists include but are not limited to cimetidine, famotidine, nizatidine, ranitidine, roxatidine, ebrotidine, lafutidine, niperotidine, potentidine, and zolantidine.

Mast cell stabilizers include but are not limited to cromolyn, nedocromil, ketotifen, lodoxamide, pemirolast, olopatadine, azelastine, and epinastine. Several histamine H1 receptor antagonists are known to act as mast cell stabilizers, these include loratadine, desloratadine, fexofenadine, terfenadine, and cetirizine.

Inhibitors and substrates of cytochrome P450 isoform 2D6 (CYP2D6) include but are not limited to: codeine, dextromethorphan, quinidine, hydroxychloroquine, diphenhydramine, clemastine, tripelennamine, promethazine, hydroxyzine, chlorpheniramine, terfenadine, and cimetidine.

Antinauseants and antiemetic agents suitable for use in ACIID include but are not limited to: 5-HT3 receptor antagonists such as ondansetron, granisetron, dolasetron, palonosetron, tropisetron, ramosetron, azasetron, cilansetron, and lerisetron; anticholinergics such as scopolamine (hyoscine); dopamine-receptor antagonists such as chlorpromazine, prochlorperazine, perphenazine, thiethylperazine, fluphenazine, metoclopramide, domperidone, haloperidol, and droperidol. Certain histamine H1 receptor antagonists are also effective antinauseants, these include but are not limited to: promethazine, cyclizine, hydroxyzine, diphenhydramine, dimenhydrinate, and meclizine.

Opioid antagonists useful in the methods of the present invention include, but are not limited to naloxone, naltrexone, nalmefene, alvimopan, methylnaltrexone, N-methylnaloxone (naloxone methiodide), diprenorphine, naloxonazine, naloxone benzoylhydrazone, and N-methylnalmefene.

Any agent used in ACIID may be administered via any of several routes, including but not limited to oral, intravenous, intramuscular, subcutaneous, intralingual, sublingual, transmucosal, transdermal, intranasal, inhalation, intrarectal, or intravaginal routes. Formulations may be immediate-release, delayed-release, extended-release, sustained-release, or any combination thereof.

### Administration of Drugs

When administered, the pharmaceutical preparations of the invention are applied in pharmaceutically acceptable compositions. Such preparations may routinely contain salts, buffering agents, preservatives, compatible carriers, lubricants and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluenesulfonic, tartaric, citric, methanesulfonic, formic, succinic, naphthalene-2-sulfonic, pamoic, 3-hydroxy-2-naphthalenecarboxylic, and benzene sulfonic.

The pharmaceutical preparations of the present invention may include or be diluted into a pharmaceutically-acceptable carrier. The term “pharmaceutically-acceptable carrier” as used herein means one or more compatible solid or liquid filler, diluents or encapsulating substances which are suitable for administration to a human or other mammal such as a dog, cat, horse, cow, sheep, or goat. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carrier formulations suitable for oral administration, for suppositories, and for parenteral administration, etc., can be found in Remington's Pharmaceutical Sciences, Mack Publishing Company, Easton, Pa.

The pharmaceutical preparations of the invention, as well as the pharmaceutical preparations that are administered to treat IBD, are free of bioavailable calcium and bioavailable calcium salts. “Free of calcium,” as used herein, means that calcium, including ions thereof, is present in the pharmaceutical preparation in a concentration of 1% or less. In some embodiments, there may be less than 0.5%, 0.1%, 0.01%, 0.001%, and even 0.0001%. Preferably, there is no detectable level of calcium present. In particular, the pharmaceutical preparations of the present invention are free of exogenously or intentionally added bioavailable calcium and bioavailable calcium salts such as soluble calcium salts including ascorbate, gluconate, glucoheptonate, dobesilate, glucobionate, levulinate, lactate, lactobionate, pantotenate, ketoglutarate, borogluconate, and the like.

Aqueous formulations may include one or more of a chelating agent, a buffering agent, an anti-oxidant, an isotonicity agent, and a preservative. In the case of quaternary amine derivatives of noroxymorphone, a chelating agent can be added and pH can be adjusted to between 3.0 and 3.5. Preferred such formulations that are stable to autoclaving and long term storage are described in co-pending application Ser. No. 60/461,611, filed on the same date hereof, entitled “Pharmaceutical Formulation”, the disclosure of which is incorporated herein by reference.

Chelating agents include: ethylenediaminetetraacetic acid (EDTA) and derivatives thereof, citric acid and derivatives thereof, niacinamide and derivatives thereof, sodium desoxycholate and derivatives thereof.

Buffering agents include: citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate and benzoic acid, and combinations thereof.

Antioxidants include: those selected from the group consisting of an ascorbic acid derivative, butylated hydroxy anisole, butylated hydroxy toluene, alkyl gallate, sodium meta-bisulfite, sodium bisulfite, sodium dithionite, sodium thioglycollate, sodium formaldehyde sulfoxylate, tocopheral and derivatives thereof, monothioglycerol, and sodium sulfite. The preferred antioxidant is monothioglycerol.

Isotonicity agents include: those selected from the group consisting of sodium chloride, mannitol, lactose, dextrose, glycerol, and sorbitol.

Preservatives that can be used with the present compositions include benzyl alcohol, parabens, thimerosal, chlorobutanol and benzalkonium chloride and preferably benzalkonium chloride is used. Typically, the preservative will be present in a composition in a concentration of up to about 2% by weight. The exact concentration of the preservative, however, will vary depending upon the intended use and can be easily ascertained by one skilled in the art.

Any of the active agents (i.e., ingredients) may be provided in particles. Particles as used herein means nano or microparticles (or in some instances larger) which consist in whole or in part of the peripheral opioid antagonists or other therapeutic agent(s) as described herein. The particles may contain the active ingredients in a core surrounded by a coating, including, but not limited to, an enteric coating. The active ingredients also may be dispersed throughout the particles. The active ingredients also may be adsorbed into the particles. The particles may be of any order release kinetics, including zero order release, first order release, second order release, delayed release, sustained release, immediate release, and any combination thereof, etc. The particle may include, in addition to the active ingredients, any of those materials routinely used in the art of pharmacy and medicine, including, but not limited to, erodible, nonerodible, biodegradable, or nonbiodegradable material or combinations thereof. The particles may be microcapsules which contain the antagonist in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as nonimmediate release formulations, with nonimmediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.” Delivery systems specific for the gastrointestinal tract are roughly divided into three types: the first is a delayed release system designed to release a drug in response to, for example, change in pH or temperature; the second is a timed-release system designed to release a drug after a predetermined time; and the third is a microflora enzyme system making use of the abundant enterobacteria in the lower part of the gastrointestinal tract.

An example of a delayed release system is one that uses, for example, an acrylic or cellulosic coating material and dissolves on pH change. Because of ease of preparation, many reports on such “enteric coatings” have been made. In general, an enteric coating is one which passes through the stomach without releasing substantial amounts of drug in the stomach (i.e., less than 10% release, 5% release and even 1% release in the stomach) and sufficiently disintegrating in the intestine tract (by contact with approximately neutral or alkaline intestine juices) to allow the transport (active or passive) of the active agent through the walls of the intestinal tract.

Various in vitro tests for determining whether or not a coating is classified as an enteric coating have been published in the pharmacopoeia of various countries. A coating which remains intact for at least 2 hours, in contact with artificial gastric juices such as HCl of pH 1 at 36 to 38° C. and thereafter disintegrates within 30 minutes in artificial intestinal juices such as a KH₂PO₄ buffered solution of pH 6.8 is one example. One such well known system is EUDRAGIT material, commercially available and reported on by Behringer, Manchester University, Saale Colo., and the like. Enteric coatings are discussed further, below.

A timed release system is represented by Time Erosion System (TES) by Fujisawa Pharmaceutical Co., Ltd. and Pulsincap by R. P. Scherer. According to these systems, the site of drug release is decided by the time of transit of a preparation in the gastrointestinal tract. Since the transit of a preparation in the gastrointestinal tract is largely influenced by the gastric emptying time, some time release systems are also enterically coated.

Systems making use of the enterobacteria can be classified into those utilizing degradation of azoaromatic polymers by an azo reductase produced from enterobacteria as reported by the group of Ohio University (M. Saffran et al., Science, Vol. 233: 1081 (1986)) and the group of Utah University (J. Kopecek et al., Pharmaceutical Research, 9(12), 1540-1545 (1992)); and those utilizing degradation of polysaccharides by beta-galactosidase of enterobacteria as reported by the group of Hebrew University (unexamined published Japanese patent application No. 5-50863 based on a PCT application) and the group of Freiberg University (K. H. Bauer et al., Pharmaceutical Research, 10(10), S218 (1993)). In addition, the system using chitosan degradable by chitosanase by Teikoku Seiyaku K. K. (unexamined published Japanese patent application No. 4-217924 and unexamined published Japanese patent application No. 4-225922) is also included.

The enteric coating is typically although not necessarily a polymeric material. Preferred enteric coating materials comprise bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers such as cellulose acetate phthalate, cellulose acetate trimellitate, hydroxypropylmethyl cellulose phthalate, hydroxypropyhnethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl methacrylate (e.g., those copolymers sold under the tradename “EUDRAGIT”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Combinations of different coating materials may also be used. Well known enteric coating material for use herein are those acrylic acid polymers and copolymers available under the tradename EUDRAGIT from Rohm Pharma (Germany). The EUDRAGIT series E, L, S, RL, RS and NE copolymers are available as solubilized in organic solvent, as an aqueous dispersion, or as a dry powder. The EUDRAGIT series RL, NE, and RS copolymers are insoluble in the gastrointestinal tract but are permeable and are used primarily for extended release. The EUDRAGIT series E copolymers dissolve in the stomach. The EUDRAGIT series L, L-30D and S copolymers are insoluble in stomach and dissolve in the intestine, and are thus most preferred herein.

A particular methacrylic copolymer is EUDRAGIT L, particularly L-30D and EUDRAGIT L100-55. In EUDRAGIT L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particular methacrylic acid polymer is EUDRAGIT S, which differs from EUDRAGIT L-30D in that the ratio of free carboxyl groups to ester groups is approximately 1:2. EUDRAGIT S is insoluble at pH below 5.5, but unlike EUDRAGIT L-30D, is poorly soluble in gastrointestinal fluids having a pH in the range of 5.5 to 7.0, such as in the small intestine. This copolymer is soluble at pH 7.0 and above, i.e., the pH generally found in the colon. EUDRAGIT S can be used alone as a coating to provide drug delivery in the large intestine. Alternatively, EUDRAGIT S, being poorly soluble in intestinal fluids below pH 7, can be used in combination with EUDRAGIT L-30D, soluble in intestinal fluids above pH 5.5, in order to provide a delayed release composition which can be formulated to deliver the active agent to various segments of the intestinal tract. The more EUDRAGIT L-30D used, the more proximal release and delivery begins, and the more EUDRAGIT S used, the more distal release and delivery begins. It will be appreciated by those skilled in the art that both EUDRAGIT L-30D and EUDRAGIT S can be replaced with other pharmaceutically acceptable polymers having similar pH solubility characteristics.

In certain embodiments of the invention, the preferred enteric coating is ACRYL-EZE™ (methacrylic acid copolymer type C; Colorcon, West Point, Pa.).

The enteric coating provides for controlled release of the active agent, such that drug release can be accomplished at some generally predictable location. The enteric coating also prevents exposure of the therapeutic agent and carrier to the epithelial and mucosal tissue of the buccal cavity, pharynx, esophagus, and stomach, and to the enzymes associated with these tissues. The enteric coating therefore helps to protect the active agent, carrier and a patient's internal tissue from any adverse event prior to drug release at the desired site of delivery. Furthermore, the coated material of the present invention allow optimization of drug absorption, active agent protection, and safety. Multiple enteric coatings targeted to release the active agent at various regions in the gastrointestinal tract would enable even more effective and sustained improved delivery throughout the gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to particles of the therapeutic agent(s), tablets of the therapeutic agent(s), capsules containing the therapeutic agent(s) and the like, using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman, et al. (New York: Marcel Dekker, Inc., 1989), and in Ansel, et al., Pharmaceutical Dosage Forms and Drug Delivery Systems, 6^{th} Ed. (Media, Pa.: Williams & Wilkins, 1995). The coating thickness, as noted above, must be sufficient to ensure that the oral dosage form remains intact until the desired site of topical delivery in the lower intestinal tract is reached.

In another embodiment, drug dosage forms are provided that comprise an enterically coated, osmotically activated device housing a formulation of the invention. In this embodiment, the drug-containing formulation is encapsulated in a semipermeable membrane or barrier containing a small orifice. As known in the art with respect to so-called “osmotic pump” drug delivery devices, the semipermeable membrane allows passage of water in either direction, but not drug. Therefore, when the device is exposed to aqueous fluids, water will flow into the device due to the osmotic pressure differential between the interior and exterior of the device. As water flows into the device, the drug-containing formulation in the interior will be “pumped” out through the orifice. The rate of drug release will be equivalent to the inflow rate of water times the drug concentration. Suitable materials for the semipermeable membrane include, but are not limited to, polyvinyl alcohol, polyvinyl chloride, semipermeable polyethylene glycols, semipermeable polyurethanes, semipermeable polyamides, semipermeable sulfonated polystyrenes and polystyrene derivatives; semipermeable poly(sodium styrenesulfonate), semipermeable poly(vinylbenzyltrimethylammonium chloride), and cellulosic polymers such as cellulose acetate, cellulose diacetate, cellulose triacetate, cellulose propionate, cellulose acetate propionate, cellulose acetate butyrate, cellulose trivalerate, cellulose trilmate, cellulose tripalmitate, cellulose trioctanoate, cellulose tripropionate, cellulose disuccinate, cellulose dipalmitate, cellulose acetate succinate, cellulose propionate succinate, cellulose acetate octanoate, cellulose valerate palmitate, cellulose acetate heptanate, cellulose acetaldehyde dimethyl acetate, cellulose acetate ethylcarbamate, cellulose acetate methylcarbamate, cellulose dimethylaminoacetate and ethylcellulose.

Enterically coated, osmotically activated devices can be manufactured using conventional materials, methods and equipment. For example, osmotically activated devices may be made by first encapsulating, in a pharmaceutically acceptable soft capsule, a liquid or semi-solid formulation as described previously. This interior capsule is then coated with a semipermeable membrane composition (comprising, for example, cellulose acetate and polyethylene glycol 4000 in a suitable solvent such as a methylene chloride-methanol admixture), for example using an air suspension machine, until a sufficiently thick laminate is formed, e.g., around 0.05 mm. The semipermeable laminated capsule is then dried using conventional techniques. Then, an orifice having a desired diameter (e.g., about 0.99 mm) is provided through the semipermeable laminated capsule wall, using, for example, mechanical drilling, laser drilling, mechanical rupturing, or erosion of an erodible element such as a gelatin plug. The osmotically activated device may then be enterically coated as previously described. For osmotically activated devices containing a solid carrier rather than a liquid or semi-solid carrier, the interior capsule is optional; that is, the semipermeable membrane may be formed directly around the carrier-drug composition. However, preferred carriers for use in the drug-containing formulation of the osmotically activated device are solutions, suspensions, liquids, immiscible liquids, emulsions, sols, colloids, and oils. Particularly preferred carriers include, but are not limited to, enterically coated capsules containing liquid or semisolid drug formulations.

In another embodiment, drug dosage forms are provided that comprise a sustained release coated device housing a formulation of the invention. In this embodiment, the drug-containing formulation is encapsulated in a sustained release membrane. The membrane may be semipermeable, as described above. Semipermeable membranes allow passage of water inside the coated device and then dissolve the drug. The dissolved drug solution then diffuses out through the semipermeable membrane. The rate of drug release therefore depends upon the thickness of the coated film and the release of drug can begin in any part of the GI tract. Suitable membrane materials include ethyl cellulose.

In another embodiment, drug dosage forms are provided that comprise a sustained release device housing a formulation of the invention. In this embodiment, the drug-containing formulation is uniformly mixed with a sustained release polymer. These sustained release polymers may be high molecular weight water-soluble polymers, which when contacted may be water, swell and create channels for water to diffuse inside and dissolve the drug. As the polymers swell and dissolve in water, more of drug is exposed to water for dissolution. Such a system is generally referred to as a sustained release matrix. Suitable materials for such a system include hydropropyl methylcellulose, hydroxypropyl cellulose, hydroxyethyl cellulose, and methyl cellulose.

In another embodiment, drug dosage forms are provided that comprise an enteric coated device housing a sustained release formulation of the invention. In this embodiment, the drug containing product described above coated with an enteric polymers. Such a device does not release any drug in the stomach. When the device reaches the intestine, the enteric polymer begins to dissolve and release the drug. The drug release may take place in a sustained release fashion.

Cellulose coatings include those of cellulose acetate phthalate and trimellitate; methacrylic acid copolymers, e.g. copolymers derived from methylacrylic acid and esters thereof, containing at least 40% methylacrylic acid; and especially hydroxypropyl methylcellulose phthalate. Methylacrylates include those of molecular weight above 100,000 daltons based on, e.g. methylacrylate and methyl or ethyl methylacrylate in a ratio of about 1:1. Typical products include EUDRAGIT L, e.g. L 100-55, marketed by Rohm GmbH, Darmstadt, Germany. Typical cellulose acetate phthalates have an acetyl content of 17-26% and a phthalate content of from 30-40% with a viscosity of ca. 45-90 cP. Typical cellulose acetate trimellitates have an acetyl content of 17-26%, a trimellityl content from 25-35% with a viscosity of ca. 15-20 cS. An example of a cellulose acetate trimellitate is the marketed product CAT (Eastman Kodak Company, USA). Hydroxypropyl methylcellulose phthalates typically have a molecular weight of from 20,000 to 130,000 daltons, a hydroxypropyl content of from 5 to 10%, a methoxy content of from 18 to 24% and a phthalyl content from 21 to 35%. An example of a cellulose acetate phthalate is the marketed product CAP (Eastman Kodak, Rochester N.Y., USA). Examples of hydroxypropyl methylcellulose phthalates are the marketed products having a hydroxypropyl content of from 6-10%, a methoxy content of from 20-24%, a phthalyl content of from 21-27%, a molecular weight of about 84,000 daltons, known under the trade mark HP50 and available from Shin-Etsu Chemical Co. Ltd., Tokyo, Japan, and having a hydroxypropyl content, a methoxyl content, and a phthalyl content of 5-9%, 18-22% and 27-35%, respectively, and a molecular weight of 78,000 daltons, known under the trademark HP55 and available from the same supplier.

The therapeutic agents may be provided in capsules, coated or not. The capsule material may be either hard or soft, and as will be appreciated by those skilled in the art, typically comprises a tasteless, easily administered and water soluble compound such as gelatin, starch or a cellulosic material. The capsules are preferably sealed, such as with gelatin bands or the like. See, for example, Remington: The Science and Practice of Pharmacy, Nineteenth Edition (Easton, Pa.: Mack Publishing Co., 1995), which describes materials and methods for preparing encapsulated pharmaceuticals.

The therapeutic agents may be provided in suppositories. Suppositories are solid dosage forms of medicine intended for administration via the rectum. Suppositories are compounded so as to melt, soften, or dissolve in the body cavity (around 98.6 F.) thereby releasing the medication contained therein. Suppository bases should be stable, nonirritating, chemically inert, and physiologically inert. Many commercially available suppositories contain oily or fatty base materials, such as cocoa butter, coconut oil, palm kernel oil, and palm oil, which often melt or deform at room temperature necessitating cool storage or other storage limitations. U.S. Pat. No. 4,837,214 to Tanaka, et al. describes a suppository base comprised of 80 to 99 percent by weight of a lauric-type fat having a hydroxyl value of 20 or smaller and containing glycerides of fatty acids having 8 to 18 carbon atoms combined with 1 to 20 percent by weight diglycerides of fatty acids (which erucic acid is an example of). The shelf life of these type of suppositories is limited due to degradation. Other suppository bases contain alcohols, surfactants, and the like which raise the melting temperature but also can lead to poor absorption of the medicine and side effects due to irritation of the local mucous membranes (see for example, U.S. Pat. No. 6,099,853 to Hartelendy et al., U.S. Pat. No. 4,999,342 to Ahmad, et al., and U.S. Pat. No. 4,765,978 to Abidi, et al.).

The base used in the pharmaceutical suppository composition of this invention include, in general, oils and fats comprising triglycerides as main components such as cacao butter, palm fat, palm kernel oil, coconut oil, fractionated coconut oil, lard and WITEPSOL® waxes such as lanolin and reduced lanolin; hydrocarbons such as VASELINE®., squalene, squalane and liquid paraffin; long to medium chain fatty acids such as caprylic acid, lauric acid, stearic acid and oleic acid; higher alcohols such as lauryl alcohol, cetanol and stearyl alcohol; fatty acid esters such as butyl stearate and dilauryl malonate; medium to long chain carboxylic acid esters of glycerin such as triolein and tristearin; glycerin-substituted carboxylic acid esters such as glycerin acetoacetate; and polyethylene glycols and its derivatives such as macrogols and cetomacrogol. They may be used either singly or in combination of two or more. If desired, the composition of this invention may further include a surface active agent, a coloring agent, etc., which are ordinarily used in suppositories.

The pharmaceutical composition of this invention may be prepared by uniformly mixing predetermined amounts of the active ingredient, the absorption aid and optionally the base, etc. in a stirrer or a grinding mill, if required at an elevated temperature. The resulting composition may be formed into a suppository in unit dosage form by, for example, casting the mixture in a mold, or by forming it into a gelatin capsule using a capsule filling machine.

The compositions according to the present invention also can be administered as a nasal spray, nasal drop, suspension, gel, ointment, cream or powder. The administration of a composition can also include using a nasal tampon or a nasal sponge containing a composition of the present invention.

The nasal delivery systems that can be used with the present invention can take various forms including aqueous preparations, non-aqueous preparations and combinations thereof. Aqueous preparations include, for example, aqueous gels, aqueous suspensions, aqueous liposomal dispersions, aqueous emulsions, aqueous microemulsions and combinations thereof. Non-aqueous preparations include, for example, non-aqueous gels, non-aqueous suspensions, non-aqueous liposomal dispersions, non-aqueous emulsions, non-aqueous microemulsions and combinations thereof. The various forms of the nasal delivery systems can include a buffer to maintain pH, a pharmaceutically acceptable thickening agent and a humectant. The pH of the buffer can be selected to optimize the absorption of the therapeutic agent(s) across the nasal mucosa.

With respect to the non-aqueous nasal formulations, suitable forms of buffering agents can be selected such that when the formulation is delivered into the nasal cavity of a mammal, selected pH ranges are achieved therein upon contact with, e.g., a nasal mucosa. In the present invention, the pH of the compositions should be maintained from about 2.0 to about 6.0. It is desirable that the pH of the compositions is one which does not cause significant irritation to the nasal mucosa of a recipient upon administration.

The viscosity of the compositions of the present invention can be maintained at a desired level using a pharmaceutically acceptable thickening agent. Thickening agents that can be used in accordance with the present invention include methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired. Such agents can also be used in a powder formulation discussed above.

Accordingly, the invention provides a method of treating/managing diarrhea comprising administering to an individual in need thereof, a first composition, in a pharmaceutically acceptable carrier, comprising an effective amount of opioid agonist to suppress gut motility and secretion, and after a desired time interval, administering to the individual a second composition, in a pharmaceutically acceptable carrier, comprising an opioid receptor antagonist in sufficient amount to reverse the effects of the opioid agonist. The diarrhea is preferably chronic. In one embodiment, the diarrhea is “uncontrollable”, for example, when one has 5-40 bowel movements a day. With the exception of newborns, who can have stool 8-10 times a day, a child or adult typically is experiencing diarrhea if they experience more than 4 bowel movements a day. Alternatively, the diarrhea is uncontrollable when the patient cannot predict the bowel movements and is suffering from fecal incontinence.

In one embodiment, the opioid agonist is a non-receptor agonist.

In one embodiment, the desired time interval between administering the first composition and the second composition is about 6-12 hours, the time interval can be as few as 2, 3, 4, or five hours and is preferably not more than 24 hours. The time interval can be about 6, 7, 8, 9, 10, 11, 12, 13, 14, 15, 16, 17, 18, 19, 20, 21, 22, 23, or 24 hours.

The treatment can be repeated a number of times so that one administers the first pharmaceutical composition, then, after a desired time interval, the second pharmaceutical composition, then again the first pharmaceutical composition and so forth. The method can be repeated for the entire duration of the disease. For example, in IBD or IBS, the patient suffering from diarrhea during the disease flares can use the method during the entirely of the flare.

In one embodiment, the method further comprises administering the individual an additional composition comprising an agent capable of suppressing mast cell activation and degranulation. The additional agent can be added to the first composition or can be administered prior to, simultaneously with or after the administration of the first compound.

The invention also provides a method of treating diarrhea comprising administering to an individual in need thereof a first composition, in a pharmaceutically acceptable carrier, comprising an effective amount of opioid agonist to suppress gut motility and secretion, and a second composition comprising and agent capable of suppressing mast cell activation and/or degranulation.

Any of the methods described herein and throughout the specification can be used to treat diseases that involve diarrhea, particularly chronic diarrhea, specifically chronic severe diarrhea that is relatively uncontrollable by the patient. Accordingly, the methods and uses of the invention can be used for treatment/management of diarrhea caused by diseases such as inflammatory bowel disease (IBS), Crohn's disease, chronic ulcerative colitis, gastritis, collagenous colitis, irritable bowel syndrome (IBD), and chronic inflammatory duodenal bowel disorders.

In one preferred embodiment, the diarrhea is caused by IBD.

In another embodiment, the diarrhea is classified as severe diarrhea.

In another embodiment, the invention provides for use of a first pharmaceutical composition and a second pharmaceutical composition for treatment of diarrhea, wherein the first pharmaceutical composition comprises an effective amount of opioid agonist to suppress gut motility and secretion, and the second pharmaceutical composition comprises an opioid receptor antagonist in sufficient amount to reverse the effects of the opioid agonist, and wherein the second pharmaceutical composition is administered after a desired time interval after administration of the first pharmaceutical composition. The time interval can be 2, 3, 4, 5, 6, 7, 8, 9, 10, 11, 12, 13, 14, 15, 16, 17, 18, 19, 20, 21, 22, 23, or 24 hours. In one preferred embodiment, the time interval is about 6-10 hours.

In one embodiment, a further third composition is administered in addition to the first and second composition, wherein the third pharmaceutical composition comprises an effective amount of an agent capable of suppressing mast cell activation and/or degranulation. In one embodiment, the third composition is combined with the first composition. In another embodiment, the third composition is administered substantially simultaneously with the first composition. In another embodiment, the third composition is administered prior to or after administering the first composition. The purpose of the third composition is to alleviate the side effects caused by the first composition and thus a skilled artisan would be able to estimate when it would be convenient to administer the third composition. The third composition can also be administered after the first occurrence of a side effect, such as nausea. In situations, where the individual is aware of having side effects from the first composition, the third composition is preferably administered prior to or simultaneously with the first composition.

In one embodiment, the invention provides a use of a pharmaceutical composition for treatment of diarrhea, wherein the pharmaceutical composition comprises an opioid receptor antagonist in sufficient amount to reverse the effects of the opioid agonist formulated in a fast release format and an opioid receptor antagonist in sufficient amount to reverse the effects of the opioid agonist formulated in a delayed release format.

In one example, loperamide (12 mg) is administered orally in a single dose, preferably in a liquid formulation. After 8 hours, a single dose of oral naltrexone (8 mg) is administered to reverse the effects of the loperamide. The duration of the antagonist action of naltrexone is likely to be at least 24 hours in this embodiment, therefore daily dosing with loperamide is not possible. This combination of oral loperamide/oral naltrexone is suited for patients with a high degree of colonic inflammation.

In another example, a single dose of loperamide (8 mg) sufficient to provide antidiarrheal control is administered by intravenous, intramuscular, or subcutaneous injection. Parenteral administration of loperamide provides a much more rapid and reliable onset of antidiarrheal action than does oral administration. Patients may self-administer the injection using a prefilled device such as an autoinjector, pen injector, or syrette. After 10 hours, a single dose of oral naltrexone (10 mg) is administered to reverse the effects of the loperamide. This embodiment is suitable for use in patients who are not chronically dependent on antidiarrheal drugs, but still experience transient attacks of severe diarrhea requiring rapid control.

In another example, a single-tablet formulation containing both an antidiarrheal mu-opioid receptor agonist in addition to a opioid antagonist is created as follows: 1) the agonist is contained within a rapidly dissolving excipient, and 2) the antagonist is contained within a delayed-release matrix so that none of the antagonist will be released until 6 hours after ingestion. The tablet is formulated to contain exactly 2 mg of the agonist difenoxin and 4 mg of the antagonist nalmefene. This embodiment is useful in patients for whom dosing regimen compliance is an issue.

In another example, oral diphenoxylate is administered (17.5 mg/day) in single or divided doses for diarrheal control over an extended period of seven days. At a specific time each day, a single injection of naloxone (3 mg) is administered so as to provide total reversal of the antidiarrheal effect for at approximately 3 hours. Since diphenoxylate has a much longer duration of action than naloxone, the antidiarrheal effect will return once the naloxone has been metabolized and eliminated. A second injection of naloxone (3 mg) is administered 8 hours after the first injection. The injections of naloxone are repeated each day the diphenoxylate is administered. This embodiment is suitable for patients who have “continuous” uncontrollable diarrhea.

In another example, a transdermal patch containing loperamide is formulated so that the wearer receives a continuous dose of 6 mg/day over an extended period of seven days. At a specific time each day, a single injection of methylnaltrexone (0.5 mg/kg) is administered to reverse the effects of the loperamide for at least 10 hours. Since loperamide has a longer duration of action than methylnaltrexone, the antidiarrheal effect will return once the methylnaltrexone has been metabolized and eliminated. This embodiment is suitable for use in patients with “continuous” diarrhea who are taking opioid analgesics or patients with small-bowel inflammation in whom the absorption of oral drugs is compromised.

In another example, a single dose of loperamide (4 mg) is administered via intranasal spray. Repeated loperamide doses are administered over a period of one hour, with 20 minutes between each dose, until diarrhea is controlled. After 8 hours, a single dose of oral alvimopan (4 mg) is used to reverse the effects of the loperamide. Due to the long duration of action of alvimopan, daily dosing with loperamide in this embodiment is not possible. This embodiment is suitable for patients who are concurrently taking opioid analgesics for pain control.

In another example, at a specific time each day, a single dose of oral difenoxin (2 mg) is administered to effect control of diarrhea. Exactly 9 hours after the difenoxin dose, a single dose of methylnaltrexone (0.5 mg/kg) is administered by intranasal spray to reverse the effect of the difenoxin. This dosing regimen is repeated once per day, for five consecutive days. The short 3 hour elimination half-life of methylnaltrexone allows for daily dosing with difenoxin, since 15 hours (five half-lives) should be sufficient to completely eliminate methylnaltrexone from the systemic circulation. This embodiment is suitable for patients with mild inflammation who have fecal incontinence.

In another example, an extended-release injectable formulation of diphenoxylate that delivers a continuous dose of 7.5 mg/day over a period of ten days is administered. At a specific time each day, naloxone (3 mg) is administered via intranasal spray. Four hours after the first naloxone dose, a second 4 mg dose of naloxone is administered via intranasal spray. This naloxone dosing regimen is repeated once per day for ten days. The extremely short half-life of naloxone, approximately 60 minutes, necessitates two consecutive doses to reverse the effects of diphenoxylate for 8 hours. This embodiment is suitable for patients that have “continuous” diarrhea.

In another example, an inhaled nebulized formulation of loperamide (6 mg) is administered to achieve diarrheal control. After 10 hours, a transdermal cream containing nalmefene (4 mg in 2 ml cream base) is applied to the skin to reverse the effect of the loperamide. Two hours after the effects of loperamide have been reversed, the nalmefene cream is washed off. Due to the long duration of action of nalmefene, daily dosing with this regimen is not possible. This embodiment is suitable for patients receiving total parenteral nutrition.

In another example, a solid oral transmucosal matrix formulation of diphenoxylate (15 mg) on a handle, designed to release 2.5 mg/hour is administered by being placed between the cheek and gum. Once satisfactory diarrheal control has been achieved, the diphenoxylate matrix is removed from the patient's mouth. After 10 hours, oral methylnaltrexone (8 mg/kg) is administered to reverse the effect of the difenoxin. This embodiment is suitable for use in patients who require occasional control of diarrheal symptoms.

In another example, oral codeine phosphate (30 mg) is administered to control diarrhea. After 4 hours, oral naloxone (15 mg) is administered to reverse the gastrointestinal effects of the codeine. Since both codeine and naloxone have short elimination half-lives, long-term once-daily dosing with this regimen is possible. This embodiment is suitable for patients with severe diarrhea with a high degree of inflammation.

In another example, a transdermal cream containing loperamide (12 mg in 2.5 ml cream base) is applied to the skin to control diarrhea. After 8 hours, a transmucosal pharmaceutical chewing gum containing naloxone (10 mg), designed to release 0.75 mg/hour is alternately chewed and then held between the cheek and gingiva. The gum is chewed for 4 hours and then discarded, reversing the effect of the loperamide for approximately 6-7 hours. Due to the long duration of action of loperamide, the antidiarrheal effect will return once the naloxone has been eliminated. This embodiment is suitable for patients that have difficulty absorbing oral drugs due to impaired intestinal function.

In another example, a single rapidly-disintegrating tablet formulation containing 0.25 mg of difenoxin hydrochloride and 4.5 mg diphenhydramine hydrochloride is administered to control diarrhea. One additional tablet is administered every 20 minutes until diarrhea is controlled, up to 8 tablets. Diphenhydramine is a competitive inhibitor of cytochrome P450 2D6 that is an H1 antihistamine and antinauseant. Mild sedation can be expected due to the low diphenhydramine dose.

In another example, a rapidly-disintegrating tablet formulation containing 100 mg of racecadotril and 45 mg fexofenadine hydrochloride is administered every 8 hours to control diarrhea. Racecadotril is a specific enkephalinase inhibitor that has intestinal antisecretory activity without causing any reductions in gastrointestinal motility. The non-sedating histamine H1 receptor antagonist fexofenadine is a mast cell stabilizer that suppresses the itching that is associated with racecadotril administration.

In another example, a single rapidly-disintegrating tablet formulation containing 30 mg of codeine phosphate, 150 mg ranitidine hydrochloride, and 10 mg loratadine is administered to control diarrhea. Loratadine is a non-sedating H1 antihistamine that has mast cell stabilizing properties. In this embodiment, combined histamine H1 and H2 receptor blockade is used to counteract the mast cell degranulating effect of codeine.

In another example, fifteen ml of an oral liquid formulation containing 5 mg loperamide hydrochloride and 12.5 mg of chlorpromazine hydrochloride is used to control diarrhea. Chlorpromazine is a dopamine D2 antagonist that has H1 antihistaminic and antimuscarinic activities, providing an antinauseant/antiemetic effect, as well as some protection against the effects of elevated histamine levels. Mild sedation due to chlorpromazine can also be expected.

In another example, a single rapidly-disintegrating tablet formulation containing 8 mg of diphenoxylate hydrochloride, 5 mg desloratadine, and 8 mg ondansetron hydrochloride is used to control diarrhea. Desloratadine is a non-sedating histamine H1 receptor antagonist that also acts as a mast cell stabilizer. Ondansetron is a 5-HT3 (5-hydroxytryptamine) receptor antagonist that effectively suppresses nausea.

In another example, a single rapidly-disintegrating tablet formulation containing 1 mg difenoxin hydrochloride, 20 mg famotidine, and 15 mg promethazine hydrochloride is administered to control diarrhea. Famotidine is a histamine H2 receptor antagonist, whereas promethazine is a histamine H1 receptor antagonist that strongly suppresses nausea. In this embodiment, only two agents are used to produce combined H1 and H2 receptor blockade simultaneously with nausea suppression. Mild sedation due to promethazine can be expected.

In another example, a single rapidly-disintegrating tablet formulation containing 0.5 mg difenoxin hydrochloride, 2.5 mg diphenoxylate hydrochloride, and 2 mg clemastine fumarate is administered to control diarrhea. Clemastine is an inhibitor of cytochrome P450 2D6 that is an H1 antihistamine as well as a mast cell stabilizer. Mild sedation due to clemastine can be expected.

In another example, a single rapidly-disintegrating tablet formulation containing 0.25 mg difenoxin hydrochloride, 10 mg codeine phosphate, and 180 mg fexofenadine hydrochloride is administered to control diarrhea. Codeine is an antidiarrheal that is also a substrate of CYP2D6. Fexofenadine is a non-sedating H1 antihistamine as well as a mast cell stabilizer. Very mild sedation due to the low codeine dose may be expected.

In another example, five ml of an oral liquid formulation containing 0.25 mg difenoxin hydrochloride, 15 mg dextromethorphan hydrobromide, and 20 mg famotidine is administered to control diarrhea. Dextromethorphan is a substrate of CYP2D6, while famotidine is a H2 histamine receptor antagonist.

In another example, fifteen ml of an oral liquid formulation containing 2.5 mg diphenoxylate hydrochloride, 5 mg codeine phosphate, 7.5 mg dextromethorphan hydrobromide and 5 mg desloratadine is administered to control diarrhea. Desloratadine is an H1 receptor antagonist that is also a mast cell stabilizer, whereas codeine and dextromethorphan are both substrates of CYP2D6. Very mild sedation due to the low codeine dose may be expected.

In another example, a single rapidly-disintegrating tablet formulation containing 10 mg diphenoxylate hydrochloride, 120 mg fexofenadine hydrochloride, and 25 mg cyclizine is administered to control diarrhea. Both fexofenadine and cyclizine are H1 antihistamines. Fexofenadine is also a mast cell stabilizer, whereas cyclizine is also an antinauseant.

In another example, a single rapidly-disintegrating tablet formulation containing 4 mg loperamide hydrochloride, 5 mg diphenoxylate hydrochloride, and 25 mg meclizine hydrochloride is administered to control diarrhea. Meclizine is an H1 antihistamine as well as an antinauseant. Very mild sedation due to meclizine can be expected.

In another example, on the first day of antidiarrheal treatment, a single rapidly-disintegrating tablet formulation containing 16 mg loperamide hydrochloride and 10 mg loratadine is administered to the patient. On the second day of treatment, a single rapidly-disintegrating tablet formulation containing 10 mg loratadine and 8 mg ondansetron is administered to the patient. The long duration of action of the synthetic opioid loperamide obviates the need to administer it on the second day of treatment. Loratadine is an H1 receptor antagonist that is also a mast cell stabilizer, and ondansetron is an antinauseant.

In another example, on the first day of antidiarrheal treatment, a single rapidly-disintegrating tablet formulation containing 0.25 mg difenoxin hydrochloride, 15 mg codeine phosphate, and 150 mg fexofenadine hydrochloride is administered to the patient. On the second day of treatment, a single rapidly-disintegrating tablet formulation containing 5 mg codeine phosphate, 180 mg fexofenadine hydrochloride, and 10 mg famotidine is administered to the patient. On the third day of treatment, a single rapidly-disintegrating tablet formulation containing 210 mg fexofenadine hydrochloride, and 20 mg famotidine is administered to the patient.

Codeine, an antidiarrheal opioid that is also a substrate of CYP2D6, slows the metabolism of difenoxin so that only a small amount of codeine is needed on the second day of treatment to maintain the antidiarrheal effect. The histamine receptor antagonists fexofenadine and famotidine, used in combination, significantly attenuate the adverse effects of opioid-induced mast cell degranulation and histamine release.

In another example, a single rapidly-disintegrating tablet formulation containing 15 mg diphenoxylate hydrochloride, 2 mg clemastine fumarate, and 150 mg ranitidine hydrochloride is administered to control diarrhea. Twelve hours later, 8 mg of oral naltrexone, an opioid receptor antagonist, is administered to the patient to reverse the antidiarrheal effect of the diphenoxylate. Since naltrexone does not block mast cell degranulation and histamine release caused by diphenoxylate and its primary metabolites diphenoxylic acid and p-hydroxydiphenoxylic acid, the histamine receptor antagonists clemastine and ranitidine are used in this formulation.

In another example, on the first day of antidiarrheal treatment, a single rapidly-disintegrating tablet formulation containing 10 mg loperamide hydrochloride and 25 mg diphenhydramine hydrochloride is administered to the patient. Ten hours later, 3 mg of naloxone, an opioid receptor antagonist, is administered via intranasal spray to reverse the antidiarrheal effect of the loperamide for approximately three hours. Since loperamide has a much longer duration of action than naloxone, the antidiarrheal effect will return once the naloxone has been metabolized and eliminated. On the second day of treatment, a single rapidly-disintegrating tablet formulation containing 2 mg loperamide hydrochloride, 25 mg diphenhydramine hydrochloride, and 60 mg fexofenadine hydrochloride is administered to the patient. Ten hours later, 3 mg of naloxone is administered via intranasal spray to reverse the antidiarrheal effect of the loperamide. Since naloxone does not block opioid-induced mast cell degranulation, the antihistamine diphenhydramine and the mast cell stabilizer fexofenadine are used in this formulation.

### REFERENCES

Ashburn M A, et al., (1992) J Pain Symptom Manage 7(1):27-33.

Ashburn M A, et al., (1995) Anesthesiology 82(5):1146-1153.

Awouters F, et al., (1983) Annu Rev Pharmacol Toxicol 23:279-301.

Awouters F, et al., (1983) Annu Rev Pharmacol Toxicol 23:279-301.

Barbara G, et al., (2006) Neurogastroenterol Motil 18(1):6-17.

Barke K E, and Hough L B (1993) Life Sci 53(18):1391-1399.

Barrett K E (2004) World J Gastroenterol 10(5):617-619.

Barton E D, et al., (2002) Prehosp Emerg Care 6(1):54-58.

Barton E D, et al., (2005) J Emerg Med 29(3):265-271.

Bartus R T, et al., (2003) Neuropsychopharmacology 28(11):1973-1982.

Basilisco G, et al., (1985) Gut 26(7):700-3.

Basilisco G, et al., (1987) Dig Dis Sci 32(8):829-32.

Baydur A (2004) Chest 125(2):363-365.

Beil W J, et al., (1997) Histochem J 29(10):759-773.

Benziger D P, and Edelson J (1983) Drug Metab Rev 14(2):137-168.

Bischoff S C, et al., (1997) Dig Dis Sci 42(2):394-403.

Bischoff S C, et al., (1999) Gut 44(5):643-652.

Block S M, et al., (1977) S Afr Med J 51(16):553-554.

Blunk J A, et al., (2004) Anesth Analg 98(2):364-370, table of contents.

Botoman V A, et al., (1998) Am Fam Physician 57(1):57-68, 71-52.

Brown J W (1979) Jama 241(5):501-502.

Brunton L L, Lazo J S, and Parker K L, eds. (2005). Goodman & Gilman's The Pharmacological Basis of Therapeutics, 11th edn (New York, McGraw-Hill Medical Publishing Division).

Caldara R, et al., (1981) Gut 22(9):720-3.

Camilleri M (2004) Neurogastroenterol Motil 16 Suppl 2(1-2.

Camilleri M (2005) Neurogastroenterol Motil 17(2):157-165.

Camilleri M, and Talley N J (2004) Neurogastroenterol Motil 16(2):135-142.

Carter M J, et al., (2004) Guidelines for the management of inflammatory bowel disease in adults. Gut 53 Suppl 5:V1-16.

Cassel J A, et al., (2005) Eur J Pharmacol 520(1-3):29-36.

Cevikel et al., (2004) ANZ J Surg 74(10):900-904.

Cheskin U, et al., (1995) Drug Alcohol Depend 39(2):151-154.

Chrubasik J, et al., (1988) Br J Anaesth 61(2):228-230.

Clarke S, and Dargan P (2002) Emerg Med J 19(3):249.

Cole L, and Hanning C D (1990) J Pain Symptom Manage 5(2):118-126.

Coluzzi P H (1998) J Pain Symptom Manage 16(3):184-192.

Coluzzi P H, et al., (2001) Pain 91(1-2):123-130.

Cooper Z R, and Rose S (2000) Mt Sinai J Med 67(2):96-105.

Crowe S E, et al., (1997) Gut 41(6):785-792.

Culpepper-Morgan J A et al., (1992) Clin Pharmacol Ther 52(1):90-95.

Curtis J A, and Goel K M (1979) Arch Dis Child 54(3):222-225.

Dale O, et al., (2002) Acta Anaesthesiol Scand 46(7):759-770.

Daly J W, and Harper J (2000) Cell Mol Life Sci 57(1):149-157.

Davis G A, et al., (2004) Am J Health Syst Pharm 61(3):261-266.

De Conno F, et al., (1995) J Clin Oncol 13(4):1004-1008.

De Luca A, and Coupar IM (1996) Pharmacol Ther 69(2):103-115.

De Schepper HUet al., (2004) Neurogastroenterol Motil 16(4):383-394.

Dean R L (2005) Front Biosci 10:643-655.

Delaney C P, et al., (2005) Dis Colon Rectum 48(6):1114-1125; discussion 1125-1116; author reply 1127-1119.

Dixon R, et al., (1987) J Clin Pharmacol 27(3):233-239.

Dixon R, et al., (1986) Clin Pharmacol Ther 39(1):49-53.

Doenicke A, et al., (1995) Intravenous morphine and nalbuphine increase histamine and catecholamine release without accompanying hemodynamic changes. Clin Pharmacol Ther 58(1):81-89.

Doenicke A W, et al., (2004) Inflamm Res 53 Suppl 2:S154-158.

Duffy L C (2000) J Nutr 130(2S Suppl):432S-436S.

Ebertz J M, et al., (1986) Agents Actions 18(5-6):455-462.

Eliot L, et al., (2002) Clin Ther 24(2):260-268.

Erbil Y, et al., (1998) Acta Chir Belg 98(6):245-249.

Ericsson C D, and Johnson P C (1990) Am J Med 88(6A):10S-14S.

Farncombe M, et al., (1994) Palliat Med 8(4):306-312.

Farrell R J, and Peppercorn M A (2002) Lancet 359(9303):331-340.

Fine K D, and Schiller L R (1999) Gastroenterology 116(6):1464-1486.

Foral P A, et al., (2004) Chest 125(2):691-694.

Foss J F (2001) Am J Surg 182(5A Suppl):19S-26S.

Fox C C, et al., (1990) Gastroenterology 99(1):119-124.

Frye R F, et al., (1996) Br J Clin Pharmacol 42(3):301-306.

Gal T J, et al, (1986) Clin Pharmacol Ther 40(5):537-542.

Galligan J J, and Vanner S (2005) Neurogastroenterol Motil 17(5):643-653.

Gambling D, et al., (2005) Anesth Analg 100(4):1065-1074.

Gan S I, and Beck P L (2003) Am J Gastroenterol 98(11):2363-2371.

Gelbmann C M, et al., (1999) Gut 45(2):210-217.

Glatt M M (1972) Int J Addict 7(3):593-596.

Gonenne J, et al., (2005) Clin Gastroenterol Hepatol 3(8):784-791.

Gordon E M, et al., (2004) BMC Pharmacol 4(1):21.

Gowan J D, et al., (1988) Can J Anaesth 35(2):143-148.

Greenwood-Van Meerveld B, et al., (2004) Neurogastroenterol Motil 16 Suppl 2:46-53.

Grossmann M, et al., (1996) Clin Pharmacol Ther 60(5):554-560.

Grundy D, et al., (2004) Neurogastroenterol Motil 16 Suppl 2:29-37.

Hammell D C, et al., (2004) J Control Release 97(2):283-290.

Harper J L, et al., (1997) Proc Natl Acad Sci USA 94(26):14912-14917.

Hawkes N D, et al., (2001) Aliment Pharmacol Ther 15(5):625-630.

He S H (2004) World J Gastroenterol 10(3):309-318.

He S H, and Xie H (2004) World J Gastroenterol 10(3):332-336.

He S H, and Xie H (2004) World J Gastroenterol 10(3):337-341.

He S H, and Xie H (2004) World J Gastroenterol 10(3):323-326.

He S H, He Y S, and Xie H (2004) World J Gastroenterol 10(3):327-331.

He S H, Xie H, and He Y S (2004) World J Gastroenterol 10(3):319-322.

Heel R C, et al., (1978) Drugs 15(1):33-52.

Hendrickson B A et al., (2002) Clin Microbiol Rev 15(1):79-94.

Hermens J M, et al., (1985) Anesthesiology 62(2):124-129.

Higgins M J, et al., (1991) Anaesthesia 46(11):973-976.

Holzer P (2004) Neurosci Lett 361(1-3):192-195.

Ives T J, and Stults C C (1983) Br J Psychiatry 143:513-514.

Jackson L S, and Stafford J E (1987) J Pharmacol Methods 18(3):189-197.

Jaffe J H, et al., (1980) Clin Pharmacol Ther 28(6):812-819.

Jaiswal J, et al., (1999) Int J Pharm 179(1):129-134.

Jimenez-Lerma J Met al., (2002) Addiction 97(7):819-824.

Joshi G P, et al., (1999) Anesthesiology 90(4):1007-1011.

Kalia Y N, et al., (2004) Adv Drug Deliv Rev 56(5):619-658.

Kaplan M A, et al., (1999) Clin Pediatr (Phila) 38(10):579-591.

Karim A, et al., (1972) Clin Pharmacol Ther 13(3):407-419.

Kelly A M, and Koutsogiannis Z (2002) Emerg Med J 19(4):375.

Kelly A M, et al., (2005) Med J Aust 182(1):24-27.

Kharasch E Det al., (2004) J Clin Pharmacol 44(3):224-233.

Killinger J M, et al., (1979) J Clin Pharmacol 19(4):211-218.

Kim S, et al., (1997) J Nucl Med 38(11):1726-1731.

Kleinman M H, and Arnon D (1977) Br J Addict Alcohol Other Drugs 72(2):167-169.

Knutson L, et al., (1990) Gastroenterology 98(4):849-854.

Kornbluth A, and Sachar D B (2004) Am J Gastroenterol 99(7):1371-1385.

Kruis W (2004) Aliment Pharmacol Ther 20 Suppl 4:75-78.

Kueppers P M, et al., (1993) Ann Surg 217(3):286-292.

Latasch L, et al., (1997) [Anaesthesist 46(3):191-194.

Leslie J B (2005) Ann Pharmacother 39(9):1502-1510.

Leslie K A, et al., (1994) Can J Surg 37(2):143-147.

Lichtenstein G R, et al., (2004) Am J Gastroenterol 99(1):91-96.

Lichtman S M (2001) J Pediatr Gastroenterol Nutr 33(1):1-10.

Maio R F, et al., (1987) Ann Emerg Med 16(5):572-573.

Manatsathit S et al., (2002) J Gastroenterol Hepatol 17 Suppl:S54-71.

McCarron M M, et al., (1991) Pediatrics 87(5):694-700.

McGuire J L, et al., (1978) Arch Int Pharmacodyn Ther 236(1):51-59.

Meissner W et al., (2000) Pain 84(1):105-109.

Mellstrand T (1987) Scand J Gastroenterol Suppl. 130:65-6.

Mikus G, et al., (1997) Clin Pharmacol Ther 61(4):459-466.

Mukherjee S, et al., (2002) J Adv Nurs 37(4):355-363.

Mycyk M B, et al., (2003) J Emerg Med 24(2):185-187.

Mystakidou K, et al., (2005) Am J Hosp Palliat Care 22(3):228-232.

Niemegeers C J, et al., (1979) J Pharmacol Exp Ther 210(3):327-333.

Nieuwenhuijs V B, et al., (1998) Ann Surg 228(2):188-193.

Nieuwenhuijs V B, et al., (1999) Eur J Clin Invest 29(1):33-40.

Nishida Y, et al., (2002) Hepatogastroenterology 49(45):678-682.

Ostrop N J, et al., (1998Pharmacotherapy 18(4):863-865.

O'Sullivan M, et al., (2000) Neurogastroenterol Motil 12(5):449-457.

Palmer K R, et al., (1980) Gastroenterology 79(6):1272-1275.

Panchagnula R, et al., (2001) Int J Pharm 219(1-2):95-105.

Panchagnula R, et al., (2005) Int J Pharm 293(1-2):213-223.

Pappagallo M (2001) Am J Surg 182(5A Suppl):11S-18S.

Park J H, et al., (2006) J Gastroenterol Hepatol 21(1 Pt 1):71-78.

Paulson D M, et al., (2005) J Pain 6(3):184-192.

Pelemans W, and Vantrappen F (1976) Gastroenterology 70(6):1030-1034.

Perez-Calderon R, and Gonzalo-Garijo MA (2004) Anaphylaxis due to loperamide. Allergy 59(3):369-370.

Piercey M F, Ruwart M J (1979) Br J Pharmacol. 66(3):373-5.

Portenoy R K, et al., (1993) Anesthesiology 78(1):36-43.

Poullis A, et al., (2002) Aliment Pharmacol Ther 16(4):675-681.

Raithel M, et al., (1995) Int Arch Allergy Immunol 108(2):127-133.

Rao S S (2004) Am J Gastroenterol 99(8):1585-1604.

Ripamonti C, and Bruera E (1991) J Palliat Care 7(1):30-35.

Ripamonti C, and De Conno F (1997) J Pain Symptom Manage 13(5):250-251.

Ripamonti C, et al., (1995) Ann Oncol 6(8):841-843.

Rook E J, et al., (2006) Drug Alcohol Depend 85(3):185-190.

Rubens R, et al., (1972) Difenoxine (R 15403), the active metabolite of diphenoxylate (R 1132). 5. Clinical comparison of difenoxine and diphenoxylate in volunteers and in patients with chronic diarrhea. Double-blind cross-over assessments. Arzneimittelforschung 22(3):526-529.

Rumack B H, and Temple A R (1974) Pediatrics 53(4):495-500.

Runkel N S, et al., (1993) Dig Dis Sci 38(8):1530-1536.

Rutgeerts P (2002) J Gastroenterol Hepatol 17 Suppl:S176-185.

Rutgeerts P (2004) Aliment Pharmacol Ther 20 Suppl 4:106-110.

Rutgeerts P, et al., (2004) Gastroenterology 126(6):1593-1610.

Rutgeerts P J, and Targan S R (1999) Aliment Pharmacol Ther 13 Suppl 4:1.

Sadeque A J, et al., (2000) Clin Pharmacol Ther 68(3):231-237.

Salazar-Lindo E, et al., (2000) N Engl J Med 343(7):463-467.

Sartor R B (2004) Gastroenterology 126(6):1620-1633.

Scarlett Y (2004) Medical management of fecal incontinence.

Gastroenterology 126(1 Suppl 1):S55-63.

Schinkel A H, et al., (1996) J Clin Invest 97(11):2517-2524.

Schmidt W K (2001) Am J Surg 182(5A Suppl):27S-38S.

Schug S A, Zech D, and Grond S (1992) Drug Saf 7(3):200-213.

Schwab D, Hahn E G, and Raithel M (2002) Inflamm Res 51 Suppl 1:S33-34.

Schwab D, Hahn E G, and Raithel M (2003) Inflamm Res 52(4):142-147.

Schwagmeier R., et al., (1996) Anaesthesist 45(3):231-234.

Schwagmeier R et al., (1995) J Clin Anesth 7(2):109-113.

Siddiqi F A (2006) Method for management of diarrhea. U.S. Provisional Patent Application Ser. No. 60/784,661.

Smith G D, et al., (2002) J Adv Nurs 38(2):152-160.

Stellato C, et al., (1992) Anesthesiology 77(5):932-940.

Stoyanova, II, and Gulubova M V (2002) Acta Histochem 104(2):185-192.

Striebel H W, et al., (1992) Anesthesiology 77(2):281-285.

Striebel H W, et al., (1993) Anaesthesia 48(9):753-757.

Striebel H W, et al., (1995) Can J Anaesth 42(4):287-291.

Striebel H W, et al., (1996) Anesth Analg 83(3):548-551.

Striebel H W, et al., (1996) J Clin Anesth 8(1):4-8.

Striebel W H, et al., (1993) Anesth Analg 76(5):1047-1051.

Su C, et al., (2002) Am J Gastroenterol 97(10):2577-2584.

Sykes N P (1996) Palliat Med 10(2):135-144.

Takala A, et al., (1997) Acta Anaesthesiol Scand 41(2):309-312.

Tan-No K, et al., (2003) Eur J Pharm Sci 20(3):357-363.

Trottet L, et al., (2004) Int J Pharm 274(1-2):213-219.

Turker S, et al., (2004) Pharm World Sci 26(3):137-142.

Tuteja A K, and Rao S S (2004) Aliment Pharmacol Ther 19(8):829-840.

Vachharajani N N, et al., (1996) Clin Pharmacol Ther 60(3):283-294.

Vachharajani N N, et al., (1997) Biopharm Drug Dispos 18(3):191-202.

Van Den Eynden B, and Spaepen W (1995) Current Therapeutic Research 56(11):1132-1141.

Veien M, et al., (2000) Anesthesiology 92(4):1074-1081.

Walker A M, et al., (2004) Am J Gastroenterol 99(7):1333-1337.

Wandel C et al., (2002) Anesthesiology 96(4):913-920.

Warren D E (1996) J Pain Symptom Manage 11(6):378-387.

Wasserman G S, et al., (1975) Am Fam Physician 11(6):93-97.

Weidenhiller M, et al., (2000) Inflamm Res 49 Suppl 1:S35-36.

Wingate D., et al., (2001) Aliment Pharmacol Ther 15(6):773-782.

Winterkamp S, et al., (2002) Am J Gastroenterol 97(12):3071-3077.

Wood J D, and Galligan J J (2004) Neurogastroenterol Motil 16 Suppl 2(17-28.

Wood J D, and Galligan J J (2004) Neurogastroenterol Motil 16 Suppl 2:17-28.

Worsley M H, et al., (1990) Anaesthesia 45(6):449-451.

Xie H, and He S H (2005) World J Gastroenterol 11(19):2851-2857.

Yeo M, et al., (2003) J Paediatr Child Health 39(4):315-317.

Yigitler C, et al., (2004) J Gastroenterol Hepatol 19(10):1179-1186.

Yu J H, et al., (2004) J Pharm Biomed Anal 36(2):421-427.

Yuan C S (2004) J Support Oncol 2(2):111-117; discussion 119-122.

Yuan C S, and Foss J F (2000) Jama 284(11):1383-1384.

Yuan C S, et al., (1996) lin Pharmacol Ther 59(4):469-475.

Yuan C S, et al., (1997) Clin Pharmacol Ther 61(4):467-475.

Yuan C S, et al., (1998) Drug Alcohol Depend 52(2):161-165.

Yuan C S, et al., (1999) Pain 83(3):631-635.

Yuan C S, et al., (2000) Clin Pharmacol Ther 67(4):398-404.

Yuan C S, et al., (2000) Jama 283(3):367-372.

Yuan C S, et al., (2002) J Pharmacol Exp Ther 300(1):118-123.

Yuan C S, et al., (2005) J Clin Pharmacol 45(5):538-546.

Zempsky W T, and Ashburn M A (1998) Am J Anesthesiol 25(4):158-162.

Zhang H, et al., (2002) Clin Pharmacokinet 41(9):661-680.

Zutshi M, Hull T, and Hammel J (2006) Crohn's Disease: A patient's perspective (Abstract). Digestive Disease Week 2006.

All references disclosed herein are incorporated herein by reference.

We claim:

1. A method of treating diarrhea comprising administering to an individual in need thereof, a first composition, in a pharmaceutically acceptable carrier, comprising an effective amount of opioid agonist to suppress gut motility and secretion, and after a desired time interval, administering to the individual a second composition, in a pharmaceutically acceptable carrier, comprising an opioid receptor antagonist in sufficient amount to reverse the effects of the opioid agonist.

2. The method of claim 1, wherein the opioid agonist is a non-receptor agonist.

3. The method of claim 1 or 2, where the desired time interval between administering the first composition and the second composition is about 6-12 hours.

4. The method of claim 1 further comprising administering the individual an additional composition comprising an agent capable of suppressing mast cell activation and degranulation.

5. The method of claim 4, wherein the additional composition is administered simultaneously with the first composition.

6. The method of claim 4, wherein the additional composition is combined with the first composition prior to administration.

7. A method of treating diarrhea comprising administering to an individual in need thereof a first composition, in a pharmaceutically acceptable carrier, comprising an effective amount of opioid agonist to suppress gut motility and secretion, and a second composition comprising and agent capable of suppressing mast cell activation and/or degranulation.

8. The method of claim 1, wherein the individual is affected with a disease selected from the diseases consisting of inflammatory bowel disease, Crohn's disease, chronic ulcerative colitis, gastritis, collagenous colitis, irritable bowel syndrome, and chronic inflammatory duodenal bowel disorders.

9. The method of any of claim 1, wherein the individual is affected with a chronic diarrhea.

10. The method of claims 1 or 7, wherein the diarrhea is caused by IBD.

11. The method of any of claim 1, wherein the diarrhea is classified as severe diarrhea.

12-17. (canceled)

18. The method of claim 1, further comprising administering to the individual an additional composition comprising one or more inhibitors or substrates of cytochrome P450 2D6.

19. A method of treating diarrhea comprising administering to an individual in need thereof a first composition, in a pharmaceutically acceptable carrier, comprising an effective amount of an antidiarrheal agent, and a second composition comprising one or more inhibitors or substrates of cytochrome P450 2D6.

20. The method of claim 19, wherein the diarrhea is chronic.

21. The method of claim 19, wherein the diarrhea is associated with IBD.
